(19) United States
(12) Patent Application Publication
(10) Pub. No.: US 2024/0366446 A1
(43) Pub. Date: Nov. 7, 2024

(54) PATIENT SUPPORT APPARATUS WITH LOCKING FEATURES

(71) Applicant: Stryker Corporation, Kalamazoo, MI (US)

(72) Inventors: Sujay Sukumaran, Portage, MI (US); Madhu Thomas, London, Ontario (CA); Seyed Behrad Ghodsi, Portland, OR (US); Christian Fulljames, Grand Rapids, MI (US); Daniel Dehoog, Grand Rapids, MI (US); Berkay Güncan, Talas, Kayseri (TR); Donna-Marie Robertson, Portage, MI (US); Xianyu Shea, Vicksburg, MI (US); Paul Bradford, West Kirby, Wirral (GB); Teresa L. Miller, Plainwell, MI (US)

(21) Appl. No.: 18/573,241

(22) PCT Filed: Oct. 7, 2022
(86) PCT No.: PCT/US2022/046009

§ 371 Date: Dec. 21, 2023

Related U.S. Application Data
(60) Provisional application No. 63/255,240, filed on Oct. 13, 2021.
(60) Provisional application No. 63/294,096, filed on Dec. 28, 2021.
(60) Provisional application No. 63/359,239, filed on Jul. 8, 2022.

(51) Int. Cl. A61G 7/018; G06F 3/04847

(52) U.S. Cl. CPC A61G 7/018; A61G 2200/327; A61G 2203/16; A61G 2203/20; A61G 2203/42; G06F 3/04847

(57) ABSTRACT

A patient support apparatus, such as a bed, cot, stretcher, etc., includes a control panel having a touchscreen display adapted to display a plurality of screens thereon. A controller automatically displays a lock screen on the display after a period of non-use and prevents the user from navigating to a different screen until the user drags a slider icon across the display and thereafter continues to press down on the slider icon for a predetermined time period. The controller may alternatively, or additionally, be adapted to display a litter frame tilt angle on the lock screen. The controller may alternatively, or additionally, display a motion control screen that includes a first lock indicator when a first motion control is in a locked state, and that adds a second lock indicator when a user presses on the first motion control while it is in the locked state.

### BACKGROUND

This application claims the benefit of is related to U.S. Prov. Appl. Ser. No. 63/359,239 entitled PATIENT SUPPORT APPARATUS WITH LOCKING FEATURES, filed on Jul. 8, 2022; U.S. Prov. Appl. Ser. No. 63/294,096 entitled PATIENT SUPPORT APPARATUS WITH LOCKING FEATURES, filed on Dec. 28, 2021; and U.S. Prov. Appl. Ser. No. 63/255,240 entitled PATIENT SUPPORT APPARATUS WITH LOCKING FEATURES, filed on Oct. 13, 2021, which are commonly owned by Stryker Corporation and incorporated by reference in their entireties herein.

The present disclosure relates to patient support apparatuses, such as beds, cots, stretchers, recliners, or the like. More specifically, the present disclosure relates to patient support apparatuses that include a display, such as a touchscreen display.

Existing hospital beds and/or stretchers often include a touchscreen display for displaying various information about the patient support apparatus, including, but not limited to, one or more controls for controlling various functions of the patient support apparatus, and one or more indicators for indicating the status of different features of the patient support apparatus.

### SUMMARY

According to different embodiments, an improved patient support apparatus is provided that includes an improved graphical user interface that makes it easier for the user to operate and to understand the current operational status of the patient support apparatus. The graphical user interface may be a touchscreen on which is displayed a motion control screen that is adapted to make is easier to understand when one or more motion controls are temporarily locked out. The touchscreen may also, or alternatively, be configured to display a lock screen that displays important information to the user when the screen is in a locked state, thereby reducing the labor required for the user to see the important information. In some aspects, the important information is customizable and/or includes a current angle of the litter frame of the patient support apparatus. In some aspects, the graphical user interface includes an unlocking mechanism for removing the lock screen that is easy to understand, yet prevents inadvertent activation of the touchscreen. Still other features and aspects of the present disclosure will be apparent to those skilled in the art in light of the accompanying drawing and the following written description.

A patient support apparatus according to a first aspect of the present disclosure includes a support surface, an actuator, a control panel, a display, and a controller. The support surface is adapted to support a patient thereon. The actuator is adapted to move a component of the patient support apparatus. The control panel includes a first motion control. The controller is adapted to switch the first motion control between an active state and a locked state. When the first motion control is in the active state, the controller is adapted to activate the actuator in response to a user pressing on the first motion control, and when the actuator is in the locked state, the controller is adapted to not activate the actuator in response to the user pressing on the first motion control. The controller is further adapted to display a first lock indicator on the display when the first motion control is in the locked state and the user is not pressing on the first motion control, and to display a second lock indicator on the display when the first motion control is in the locked state and the user is pressing on the first motion control.

According to another aspect of the present disclosure, a patient support apparatus is provided that includes a support surface, a litter frame, a tilt angle sensor, a control panel, a display, and a controller. The support surface is adapted to support a patient thereon. The litter frame is adapted to support the support surface. The tilt angle sensor is adapted to sense a tilt angle of the litter frame. The controller is adapted to automatically display a lock screen on the display after a period of non-use of the control panel. The lock screen includes an unlock control and a litter frame tilt angle, and the controller is further adapted to not allow a user to navigate to another screen until the user activates the unlock control.

According to still another aspect of the present disclosure, a patient support apparatus is provided that includes a support surface, a control panel, a display, and a controller. The support surface is adapted to support a patient thereon. The controller is adapted to automatically display a lock screen on the display after a period of non-use of the control panel. The controller is further adapted to not allow a user to navigate to a screen other than the lock screen until the user drags a slider icon on the display and thereafter continues to press down on the slider icon for a predetermined time period.

According to still other aspects of the present disclosure, the controller may be adapted to display both the first lock indicator and the second lock indicator on the display when the first motion control is in the locked state and the user is pressing on the first motion control.

In some aspects, at least one of the first and second lock indicators is a lock symbol. The other one of the first and second lock indicators may be a circle positioned around a perimeter of the first motion control in some aspects.

The controller, in some aspects, is further adapted to fade the second lock indicator away after the user stops pressing on the first motion control.

In some aspects, the controller is further adapted to display both the first lock indicator and the second lock indicator adjacent the first motion control.

In some aspects, the patient support apparatus further comprises a second motion control and the controller is adapted to switch the second motion control between the active state and the locked state, to activate the actuator in a first direction in response to the user pressing on the first motion control, and to activate the actuator in a second and opposite direction in response to the user pressing on the second motion control.

The controller, in some aspects, is further adapted to display a third lock indicator on the display when the second motion control is in the locked state and the user is not pressing on the first motion control, and to display a fourth lock indicator on the display when the second motion control is in the locked state and the user is pressing on the second motion control.

The controller, in some aspects, is adapted to display both the third lock indicator and the fourth lock indicator on the display when the second motion control is in the locked state and the user is pressing on the second motion control.

The controller, in some aspects, is further adapted to display a lock screen on the display after a period non-use of the control panel.

In some aspects, the controller is further adapted to not allow the user to navigate to a screen other than the lock screen until the user drags a slider icon on the display and thereafter continues to press down on the slider icon for a predetermined time period.

The lock screen, in some aspects, includes an unlocking control and the controller is further adapted to not allow the user to navigate to another screen until the user activates the unlock control.

In some aspects, the lock screen includes no controls for controlling any movement of the patient support apparatus.

The patient support apparatus, in some aspects, also includes a litter frame adapted to support the support surface and a tilt angle sensor adapted to sense a tilt angle of the litter frame. The controller may be adapted to display a litter frame tilt angle on the lock screen.

In some aspects, the controller is adapted to display the litter frame tilt angle in a first manner when the litter frame tilt angle is zero degrees and in a second manner different from the first manner when the litter frame tilt angle is not zero degrees.

The different manner, in some aspects, includes changing a color of the litter frame tilt angle.

In some aspects, the controller is further adapted to display a concealment control adapted to selectively conceal and reveal the litter frame tilt angle.

The controller, in some aspects, is adapted to display a symbol when the litter frame tilt angle is concealed, to change a characteristic of the symbol when the litter frame tilt angle is non-zero, and to display a numeric value of the litter frame tilt angle when the litter frame tilt angle is not concealed.

The lock screen, in some aspects, includes no controls other than the concealment control and the unlock control.

In some aspects, the controller is adapted to display a lock icon on the lock screen and to animate unlocking movement of the lock icon as the user drags the slider icon on the display.

The controller, in some aspects, is further adapted to animate a circle being drawn around the slider icon when the user presses down on the slider icon for the predetermined time period.

The controller, in some aspects, is also adapted to finish animating the circle being drawn around the slider icon upon expiration of the predetermined time period.

In some aspects, the control panel of the patient support apparatus includes a motion control screen having an ornamental design comprised of all of, any portion of, and/or combination of components of, the screens shown herein in FIGS. 20-24.

In some aspects, the control panel of the patient support apparatus includes a supplemental motion control screen having an ornamental design comprised of all of, any portion of, and/or combination of components of, the screen shown herein in FIG. 25.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one embodiment of the disclosure;

FIG. 2 is a perspective view of a litter frame of the patient support apparatus;

FIG. 3 is a perspective view of a base of the patient support apparatus;

FIG. 4 is a plan view of a control panel that may be included with the patient support apparatus;

FIG. 5 is a block diagram of a control system of the patient support apparatus, as well as several external devices with which the patient support apparatus may be configured to communicate;

FIG. 6 is an illustrative lock screen displayable on the display of FIG. 4 illustrating a hidden tilt angle indicator when the tilt angle is zero degrees;

FIG. 7 is the lock screen of FIG. 6 shown with the tilt angle indicator unhidden and a tilt angle of zero degrees;

FIG. 8 is the lock screen of FIG. 6 shown with the tilt angle indicator hidden when the tilt angle is not zero degrees;

FIG. 9 is the lock screen of FIG. 6 shown with the tilt angle indicator unhidden and a non-zero tilt angle tilted in a first direction;

FIG. 10 is the lock screen of FIG. 6 shown with the tilt angle indicator unhidden and a non-zero tilt angle tilted in a second direction;

FIG. 11 is a lock screen customization screen displayable on the display of FIG. 4;

FIG. 12 is an unlock control displayable on the lock screen of FIGS. 6-10 when a user presses on the lock screen;

FIG. 13 is the unlock control of FIG. 12 shown with a gradient color change displaced from its position in FIG. 12;

FIG. 14 is the unlock control of FIG. 12 shown after the user has dragged the slider icon a first distance to the right;

FIG. 15 is the unlock control of FIG. 12 shown after the user has dragged the slider icon a second distance further to the right;

FIG. 16 is the unlock control of FIG. 12 shown after the user has dragged the slider icon a third distance all the way to the right;

FIG. 17 is the unlock control of FIG. 12 shown after the user has dragged the slider icon all the way to the right and continued to press on the slider icon for a first period of time;

FIG. 18 is the unlock control of FIG. 12 shown after the user has dragged the slider icon all the way to the right and continued to press on the slider icon for a second period of time longer than the first period of time;

FIG. 19 is the unlock control of FIG. 12 shown after the user has dragged the slider icon all the way to the right and continued to press on the slider icon for a third period of time longer than the second period of time;

FIG. 20 is an illustrative motion control screen displayable on the display of FIG. 4 that includes a plurality of motion controls, none of which have been locked out;

FIG. 21 is the motion control screen of FIG. 20 shown with head section controls locked out and no user pressing on the head section controls;

FIG. 22 is the motion control screen of FIG. 20 shown with head section controls locked out and the user pressing on the head section raising control;

FIG. 23 is the motion control screen of FIG. 20 shown with head section controls locked out in the moment immediately after the user has stopped pressing on the head section raising control;

FIG. 24 is the motion control screen of FIG. 20 shown with head section controls locked out and the user pressing on the head section lowering control;

FIG. 25 is a supplemental motion control screen that includes a plurality of additional motion controls and that may be displayed in response to a user pressing an arrow control in any of FIGS. 20-24;

FIG. 26 is another motion control screen displayable on the display of FIG. 4 that includes a plurality of motion controls, with one of the Trendelenburg controls selected but none of the other controls locked out;

FIG. 27 is the motion control screen of FIG. 26 shown with the other Trendelenburg control selected but none of the controls locked out;

FIG. 28 is the motion control screen of FIG. 26 with none of the controls selected or locked out, but illustrating some of the different configurations of two of the dynamic icons;

FIG. 29 is a motion lock screen with none of the lock controls selected;

FIG. 30 is the motion lock screen of FIG. 29 shown with the Fowler section lock control selected;

FIG. 31 is the motion lock screen of FIG. 29 shown with the gatch section lock control selected;

FIG. 32 is the motion lock screen of FIG. 29 shown with the deck height movement lock control selected;

FIG. 33 is the motion lock screen of FIG. 29 shown with the Fowler section, gatch section and height movement lock controls selected;

FIG. 34 is the motion control screen of FIG. 26 with all of controls locked out or in a lock state;

FIG. 34A is the motion control screen of FIG. 26 with all of controls locked out or in a lock state, but illustrating some of the different configurations of two of the dynamic icons;

FIG. 34B is the motion control screen of FIG. 34A with all of controls locked out or in a lock state, but illustrating another lock indicator adjacent each control;

FIG. 35 is another supplemental motion control screen that includes a plurality of additional motion controls associated with pre-set configurations and an auto contour control, which may be displayed in response to a user pressing an arrow control in any of the motion control screens of FIGS. 26-28, 34, and 34A;

FIG. 36 is another home screen illustrating the locked status of the patient support, which includes a CPR button;

FIG. 37 is the home screen of FIG. 36 with the CPR button but no indication that the patient support is locked;

FIG. 38 is an illustration of a scale control screen;

FIG. 39 is an illustration of a progress indicator screen;

FIG. 40 is a similar illustration to FIG. 38;

FIG. 41 is an illustration of a scale history screen in one format;

FIG. 42 is a similar illustration as FIG. 41;

FIG. 43 is an illustration of the scale history screen in a second format;

FIG. 44 is a similar illustration as FIG. 43;

FIG. 45 is a similar illustration as FIG. 43;

FIG. 46 is a similar illustration as FIG. 43;

FIG. 47 is a similar illustration of the scale history screen in the second format but in a compact configuration;

FIG. 48 is an illustration of a condensed scale history screen;

FIG. 49 is an illustration of an exit detection control screen;

FIG. 50 is a similar illustration to FIG. 49;

FIG. 51 is a similar illustration to FIG. 49;

FIG. 52 is a similar illustration to FIG. 49; and

FIG. 52A is a similar illustration to FIG. 52 with a modified banner, a modified color coding, and a modified icon for the low sensitivity control;

FIG. 53 is another home screen with a CPR button and vascular button;

FIG. 54 is a similar home screen to FIG. 53 with a modified vascular button selected and modified so that it changes size when selected and also illustrating the concealment control selected to show the tilt angle of the deck;

FIG. 55 is a similar view to FIG. 54 but with the modified CRP button selected;

FIG. 56 is another home screen similar to home screen 54 but with a modified CPR button modified to include an annular spinner to show progress of the selected CPR function when selected;

FIG. 57 is the home screen of FIG. 56 with the modified vascular button selected and modified to include an annular spinner to show progress of the selected vascular function;

FIG. 58 is a similar home screen to FIG. 53 with the modified vascular button selected and modified so that it changes color when selected, and also illustrating the concealment control selected to show the tilt angle of the deck;

FIG. 59 is a similar view to FIG. 58 but with the modified CRP button selected and modified so that it changes color when selected, and also illustrating the concealment control selected to show the tilt angle of the deck;

FIG. 60 is a supplemental motion control screen similar to the supplemental control screen shown in FIG. 35, with a CPR button and a vascular button both of which are colored differently than the other preset position control buttons; and

FIG. 61 is similar to FIG. 60 but without the CPR button.

### DETAILED DESCRIPTION OF THE EMBODIMENTS

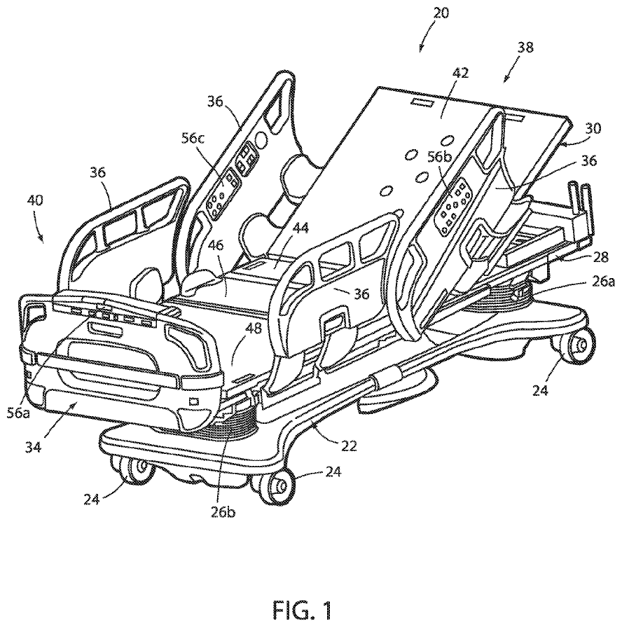

An illustrative patient support apparatus 20 that may incorporate one or more aspects of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, a residential bed, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, footboard 34, and siderails 36. Support deck 30 provides a support surface for a mattress (not shown), or other soft cushion, so that a person may lie and/or sit thereon. Support deck 30 is made of a plurality of sections, some of which are pivotable about generally horizontal pivot axes. In the embodiment shown in FIG. 1, support deck 30 includes at least a head section 42, a seat section 44, a thigh section 46, and a foot section 48, all of which generally form flat surfaces for supporting the mattress. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Notionally, the first standard width may be considered a 36 inch width, the second intermediate width may be considered a 42 inch width and the third more expanded width may be considered a 48 inch width, although these numerical widths may be varied to comprise different width values.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

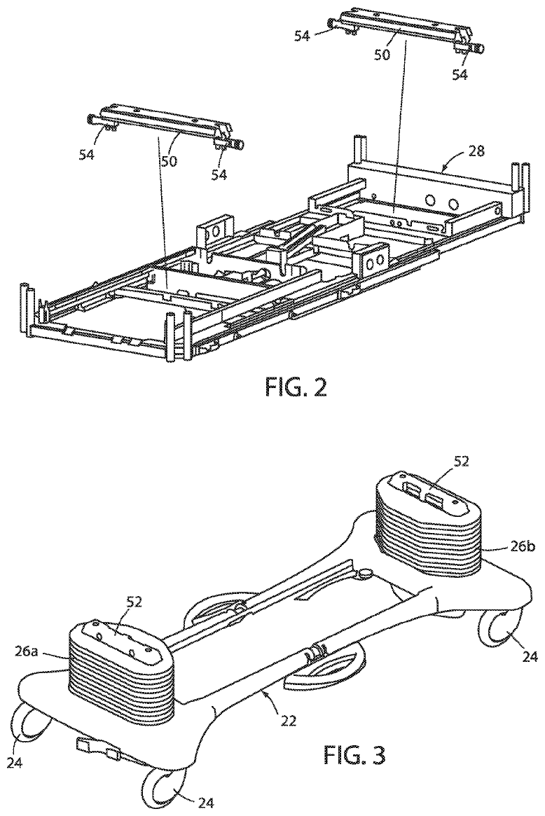

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of force sensors 54, which may be load cells, or other types of force sensors. The illustrated embodiment of patient support apparatus 20 therefore includes a total of four force sensors 54, although it will be understood by those skilled in the art that different numbers of force sensors may be used in accordance with the principles of the present disclosure. Force sensors 54 are configured to support litter frame 28. More specifically, force sensors 54 are configured such that they provide complete and exclusive mechanical support for litter frame 28 and all of the components that are supported on litter frame 28 (e.g. support deck 30, footboard 34, the headboard, siderails 36, etc.). Because of this construction, force sensors 54 are adapted to detect the weight of not only those components of patient support apparatus 20 that are supported by litter frame 28 (including litter frame 28 itself), but also any objects or persons who are wholly or partially being supported by support deck 30. The outputs of force sensors 54 are used for implementing a scale function and an exit detection function, as will be described in greater detail below.

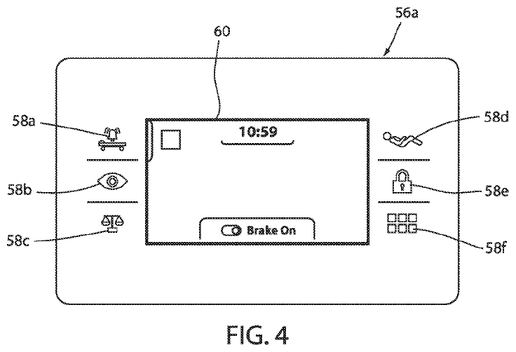

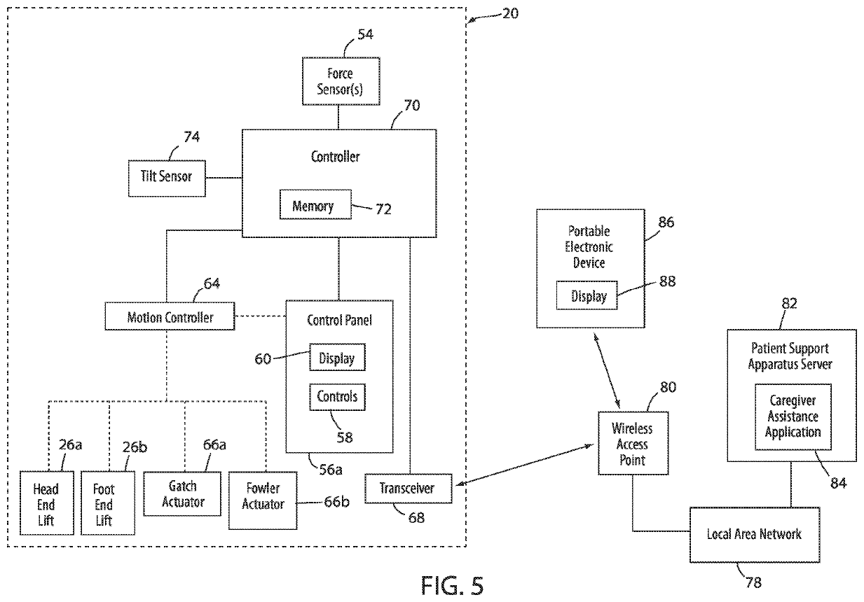

Patient support apparatus 20 further includes a plurality of control panels 56 (FIG. 1) that enable a user of patient support apparatus 20, such as a patient and/or an associated caregiver, to control one or more aspects of patient support apparatus 20. In the embodiment shown in FIG. 1, patient support apparatus 20 includes a footboard control panel 56a, a pair of outer siderail control panels 56b (only one of which is visible), and a pair of inner siderail control panels 56c (only one of which is visible). Footboard control panel 56a concealed by a lid in FIG. 1, but is otherwise visible and accessible to a caregiver when the lid is lifted. In some embodiments, the lid is eliminated from patient support apparatus 20 and the control panel 56a is always visible. Footboard control panel 56a and outer siderail control panels 56b are intended to be used by caregivers, or other authorized personnel, while inner siderail control panels 56c are intended to be used by the patient associated with patient support apparatus 20. Each of the control panels 56 includes a plurality of controls 58 (see, e.g. FIGS. 4-5), although each control panel 56 does not necessarily include the same controls and/or functionality.

Among other functions, controls 58 of control panel 56a allow a user to control one or more of the following: change a height of support deck 30, raise or lower head section 42, take patient weight readings, arm and disarm an exit detection function, activate and deactivate a brake for wheels 24, arm and disarm one or more patient support apparatus monitoring functions, change various settings on patient support apparatus 20, view the current location of the patient support apparatus 20 as determined by a location detection system, perform a calibration process on the patient support apparatus 20, and perform still other actions. One or both of the inner siderail control panels 56c also include at least one control 58 that enables a patient to call a remotely located nurse (or other caregiver).

Control panel 56a includes a display 60 (FIG. 4) configured to display a plurality of different screens thereon. Surrounding display 60 are a plurality of navigation controls 58a-f that, when activated, cause the display 60 to display different screens on display 60. For example, when a user presses navigation control 58a, control panel 56a displays an exit detection control screen on display 60 that includes one or more icons that, when touched, control an onboard exit detection function. The exit detection function is adapted to issue an alert when a patient exits from patient support apparatus 20. Such an exit detection function may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES; U.S. patent application Ser. No. 17/318,476 filed May 12, 2021, by inventors Sujay Sukumaran et al. and entitled PATIENT SUPPORT APPARATUS WITH AUTOMATIC EXIT DETECTION MODES OF OPERATION; and/or the exit detection system disclosed in commonly assigned U.S. Pat. No. 5,276,432 issued to Travis and entitled PATIENT EXIT DETECTION MECHANISM FOR HOSPITAL BED, the complete disclosures of all of which are incorporated herein by reference.

When a user presses navigation control 58b (FIG. 4), control panel 56a displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 58c, control panel 56a displays a scale control screen that includes a plurality of control icons that, when touched, control the scale system of patient support apparatus 20. The scale system of patient support apparatus 20 may take on a variety of different forms and include a variety of different features and function. In some embodiments, the scale system may include any of the same features, components, and/or and functions as the scale systems disclosed in the following commonly assigned patent references: U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES; U.S. patent application Ser. No. 63/255,211 filed Oct. 13, 2021, by inventors Sujay Sukumaran et al. and entitled PATIENT SUPPORT APPPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,33,233 issued to Michael Hayes et al. on Jun. 15, 2021, and entitled PATIENT SUPPORT APPARATUS WITH PATIENT INFORMATION SENSORS; U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG; and U.S. patent application Ser. No. 63/255,223, filed Oct. 13, 2021, by inventors Sujay Sukumaran et al. and entitled PATIENT SUPPORT APPARATUS WITH PATIENT WEIGHT MONITORING, the complete disclosures of all of which are incorporated herein by reference. The scale system may utilize the same force sensors that are utilized by the exit detection system, in some embodiments, or it may utilize one or more different sensors.

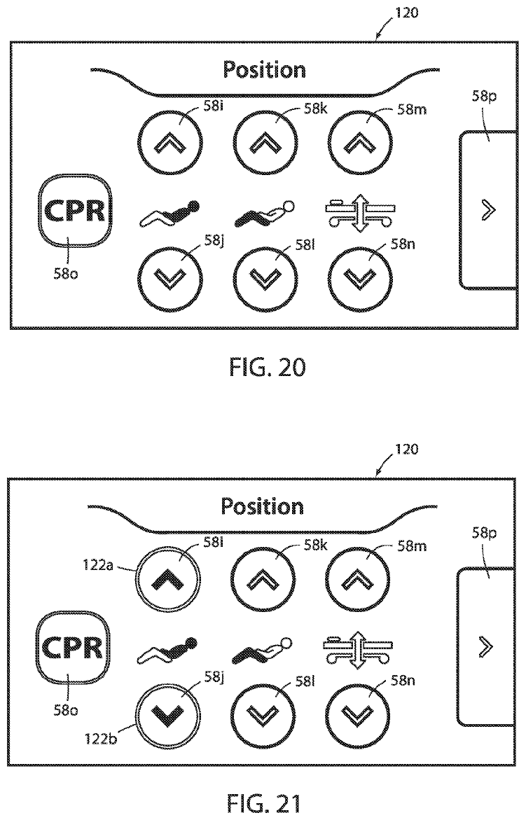

When a user presses navigation control 58d, control panel 56a displays a motion control screen that includes a plurality of control icons that, when touched, control the movement of various components of patient support apparatus 20, such as, but not limited to, the height of litter frame 28 and the pivoting of head section 42. In one embodiment, which will be discussed in greater detail below, controller 70 is configured to display a motion control screen of the type shown in FIGS. 20-24 in response to a user pressing on control 58d. In other embodiments, controller 70 may be configured to display a motion control screen in response to the user pressing control 58d that may be the same as, or similar to, the motion control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be displayed on display 60 of patient support apparatus 20.

When a user presses navigation control 58e (FIG. 4), control panel 56a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20. As will be discussed in greater detail below, patient support apparatus 20 may be configured to alter the manner in which the motion control screens of FIGS. 20-24 are displayed in response to one or more of the motion lockouts being activated.

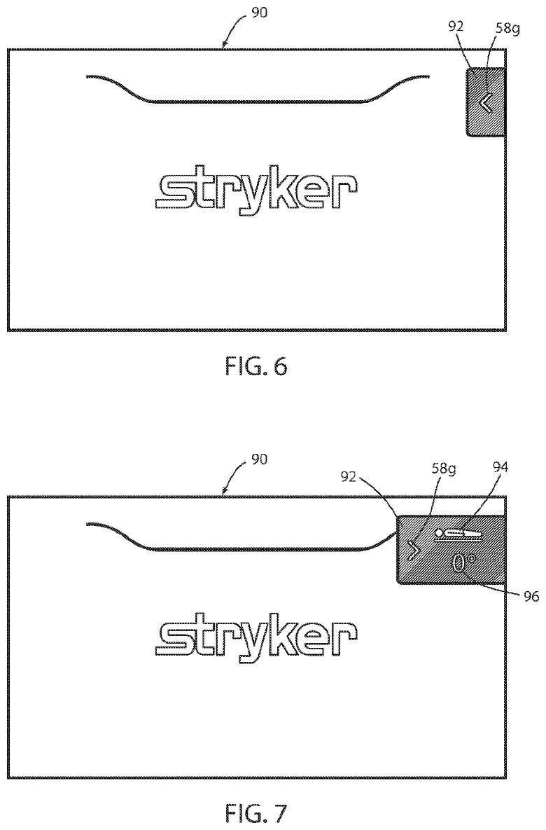

When a user presses on navigation control 58f, control panel 56a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a menu screen in shown herein in FIG. 6. Another example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 58f allows a user to navigate to a screen that enables a user to configure the communication settings between patient support apparatus 20 and one or more wall units. Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is incorporated herein by reference.

For all of the navigation controls 58a-f (FIG. 4), screens other than the ones specifically mentioned above may be displayed on display 60 in other embodiments of patient support apparatus 20 in response to a user pressing these controls. Thus, it will be understood that the specific screens mentioned above are merely representative of the types of screens that are displayable on display 60 in response to a user pressing on one or more of navigation controls 58a-f. It will also be understood that, although navigation controls 58a-f have all been illustrated in the accompanying drawings as dedicated controls that are positioned adjacent display 60, any one or more of these controls 58a-f could alternatively be touchscreen controls that are displayed at one or more locations on display 60. Still further, although controls 58a-f have been shown herein as buttons, it will be understood that any of controls 58a-f could also, or alternatively, be switches, dials, or other types of non-button controls.

As shown in FIG. 5, patient support apparatus 20 includes a control system 62 that controls various aspects of patient support apparatus 20. Control system 62 includes control panels 56 (only one of which—56a—is shown in FIG. 5), force sensors 54, a motion controller 64, lifts 26, a plurality of motorized actuators 66a-b, a network transceiver 68, a tilt sensor 74, and a main controller 70. Main controller 70 includes a memory 72. Main controller 70 and force sensors 54 together act to perform both a scale function and an exit detection function. When carrying out the exit detection function, main controller 70 and force sensors 54 are adapted to determine when an occupant leaves patient support apparatus, or moves in a way indicative of an imminent departure from the patient support apparatus. In such situations, main controller 70 is configured to issue an alert and/or notification to appropriate personnel so that proper steps can be taken in response to the occupant's departure, or imminent departure, in a timely fashion. When carrying out the scale function, the outputs of force sensors 54 are summed together to determine a patient's weight (after adjusting for the tare weight and/or other weights that may be stored in an equipment weight log).

Force sensors 54 are adapted to detect downward forces exerted by an occupant of support deck 30. Thus, when an occupant is positioned on support deck 30 and remains substantially still (i.e. not moving in a manner involving accelerations that cause forces to be exerted against support deck 30), force sensors 54 will detect the weight of the occupant (as well as the weight of any components of patient support apparatus 20 that are supported-directly or indirectly-by force sensors 54). In at least one embodiment, force sensors 54 are load cells. However, it will be understood by those skilled in the art, that force sensors 54 may be implemented as other types of sensors, such as, but not limited to, linear variable displacement transducers and/or any one or more capacitive, inductive, and/or resistive transducers that are configured to produce a changing output in response to changes in the force exerted against them.

Main controller 70 and motion controller 64 are constructed of any electrical component, or group of electrical components, that are capable of carrying out the functions described herein. In many embodiments, controllers 64 and 70 are conventional microcontrollers, although not all such embodiments need include a microcontroller. In general, controllers 64 and 70 include any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, main controller 70 and motion controller 64 are combined with each other and/or with other circuitry or controllers that are present on patient support apparatus 20. The instructions followed by controllers 64 and 70 in carrying out the functions described herein, as well as the data necessary for carrying out these functions, are stored in one or more memories that are accessible to them (e.g. memory 72 for main controller 70).

Although patient support apparatus 20 includes a total of four force sensors 54, it will be understood by those skilled in the art that different numbers of force sensors 54 may be used in accordance with the principles of the present disclosure. Force sensors 54, in at least one embodiment, are configured to support litter frame 28. When so configured, force sensors 54 are constructed to provide complete and exclusive mechanical support for litter frame 28 and all of the components that are supported on litter frame 28 (e.g. deck 30, footboard 34, and, in some embodiments, siderails 36). Because of this construction, force sensors 54 are adapted to detect the weight of not only those components of patient support apparatus 20 that are supported by the litter frame 28 (including litter frame 28 itself), but also any objects or persons who are positioned either wholly or partially on support deck 30. By knowing the weight of the components of the patient support apparatus 20 that are supported on litter frame 28, controller 70 is able to determine a tare weight that, when subtracted from a total weight sensed after a patient is supported on support deck 30, yields a patient weight.

In some embodiments, the physical location of the force sensors 54 on patient support apparatus 20 may be modified to be located on the base frame, such as shown in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. In other embodiments, the physical location of the force sensors 54 on patient support apparatus 20 may be the same as the position of the load cells disclosed in commonly assigned U.S. patent application Ser. No. 15/266,575 filed Sep. 15, 2016, by inventors Anuj Sidhu et al. and entitled PERSON SUPPORT APPARATUSES WITH EXIT DETECTION SYSTEMS, the complete disclosure of which is also incorporated herein by reference. In still other embodiments, the physical location of the force sensors 54 may be the same as the position of the load cells disclosed in U.S. Pat. No. 7,962,981 issued to Lemire et al. and entitled HOSPITAL BED, the complete disclosure of which is also incorporated herein by reference. In still other embodiments, force sensors 54 may be positioned on patient support apparatus 20 at still other locations.

Motion controller 64 (FIG. 5) is adapted to control the movement of a plurality of components of patient support apparatus 20. These components includes, but are not limited to, a head end lift or actuator 26a, a foot end lift or actuator 26b, a gatch actuator 66a, and a Fowler actuator 66b. Each of these actuators 26a-b and 66a-b may comprise a linear actuator with a motor built therein. In some embodiments, the linear actuator may be of the type disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In other embodiments, other types of powered actuators may be used, such as, but not limited to, hydraulic and/or pneumatic actuators.

In some embodiments, motion controller 64 operates in the same or similar manners to the main microcontroller 58 and its associated circuitry disclosed in commonly assigned U.S. Pat. No. 10,420,687 issued Sep. 24, 2019, to inventors Aaron Furman et al. and entitled BATTERY MANAGEMENT FOR PATIENT SUPPORT APPARATUSES, the complete disclosure of which is incorporated herein by reference. In such embodiments, motion controller 64 controls the sending of pulse width modulated (PWM) signals to the motors contained within actuators 26a-b and 66a-b, thereby controlling both the speed and the direction of movement of these actuators. Motion controller 64 may take on other forms as well.

Motion controller 64 is in communication with control panel 56 and receives signals from control panel 56 indicating when a user wishes to move one or more components of patient support apparatus 20. That is, control panel 56 includes one or more controls 58 that are adapted, when activated, to instruct motion controller 64 to carry out the desired movement of the various movable components of patient support apparatus 20, as well as one or more controls for stopping such motion. Such movement includes, but is not limited to, raising and lowering the height of litter frame 28, pivoting the Fowler section 42 up and down about a generally horizontal axis (extending laterally from one side of the patient support apparatus 20 to the other), and/or lifting and lowering a knee gatch on patient support apparatus 20. As will be discussed in greater detail, display 60 may be a touchscreen that, depending upon the particular content displayed at a particular time, includes one or more motion controls that, when activated, cause motion controller 64 to activate one or more of the actuators 26a-b and/or 66a-b.

Head end lift actuator 26a is configured to change the height of the head end 38 of litter frame 28 (e.g. the Fowler section). Foot end lift actuator 26b is configured to change the height of the foot end 40 of litter frame 28. When both of these actuators 26a and 26b are operated simultaneously and at the same speed, the height of litter frame 28 is raised or lowered without changing the general orientation of litter frame 28 with respect horizontal. When one or more of these actuators 26a and/or 26b are operated at different times and/or at different speeds, the orientation of litter frame 28 is changed with respect to horizontal. Lift actuators 26a and 26b are therefore able to tilt litter frame 28 to a variety of different orientations, including, but not limited to, a Trendelenburg orientation and a reverse-Trendelenburg orientation.

Gatch actuator 66a is adapted to raise and lower the joint that couples together the thigh section 46 and the foot section 48 of support deck 30, thereby raising and lowering the portion of the support deck 30 that is positioned close to the patient's knees. Fowler actuator 66b is adapted to raise and lower the head section (or Fowler section) 42 of the support deck 30.

Tilt angle sensor 74 is adapted to detect any tilting of litter frame 28 from a horizontal orientation. That is, tilt angle sensor 74 is adapted to detect when the litter frame 28 is tilted in a manner in which its head end 38 is higher than (or lower than) its foot end 40. In other words, tilt angle sensor 74 detects when litter frame 28 pivots about a horizontal axis that extends in a direction transverse to the longitudinal direction. In some embodiments, tilt sensor 74 includes one or more accelerometers that are used to detect the tilting of litter frame 28. The outputs from tilt sensor 74 are used by controller 70 to adjust the outputs from the force sensors 54, in at least some embodiments. This is done because, in some embodiments, force sensors 54 are only able to accurately detect purely vertical forces, and when litter frame 28 is tilted, it may exert non-vertical force components onto the force sensors 54 that are not otherwise accurately detectable by the force sensors 54. The outputs of the tilt sensor 74 may also be displayed on a lock screen (and/or another screen) on display 60 so that the caregiver is apprised of the amount of angular tilt of the litter frame 28, as will be discussed in greater detail below.

Control panel 56 (FIG. 4) communicates with main controller 70 and is adapted to allow a user to control the various functions performed by main controller 70, as well as to navigate through various screens that are displayable on display 60. The plurality of controls 58 of control panel 56 may be implemented as buttons, dials, switches, icons on a touchscreen, or other devices. Display 60 of control panel 56 may be a touchscreen that displays one or more controls 58 and/or one or more control screens, some of which are discussed in greater detail below. Display 60 may comprise an LED display, OLED display, or another type of display.

Control system 62 may include additional components beyond those shown in FIG. 5. Alternatively, or additionally, one or more of the components shown in FIG. 5 may be omitted from patient support apparatus 20, or replaced by other components.

Network transceiver 68 (FIG. 5) is adapted to communicate with a hospital's local area network 78. In some embodiments, network transceiver 68 is a conventional WiFi transceiver (i.e. IEEE 802.11 . . . ) adapted to wirelessly communicate with one or more wireless access points 80 of a hospital's local area network 78. In other embodiments, network transceiver 68 may be a wireless transceiver that uses conventional 5G technology to communicate with network 78, one or more servers hosted thereon, and/or other devices. In some embodiments, network transceiver 68 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In other embodiments, network transceiver 68 may be a conventional Ethernet transceiver electrically coupled to a conventional Ethernet port (i.e. RJ-45 jack, or the like) built into patient support apparatus 20 that allows a conventional Ethernet cable to be coupled to the patient support apparatus 20. In these embodiments, patient support apparatuses 20 may be coupled to the hospital's local area network 78 by a wired connection. In still other embodiments, patient support apparatus 20 may have both wired and wireless transceivers 68. Still further, in some embodiments, transceiver 68 may take on yet a different form (e.g. a wireless ZigBee transceiver, a Bluetooth transceiver, etc.).

Patient support apparatus 20 uses transceiver 68 (FIG. 5), in some embodiments, to communicate with a patient support apparatus server 82. Patient support apparatus server 82 may be adapted to receive status information from patient support apparatuses 20 and distribute that information to one or more other servers and/or other devices coupled to local area network 78. In at least one embodiment, patient support apparatus server 82 includes a caregiver assistance software application 84 that is adapted to communicate information between both patient support apparatuses 20 and one or more portable electronic devices 86. The portable electronic devices 86 includes, but are not limited to, smart phones, tablets, laptops, Computers on Wheels (COWs), and the like. Each portable electronic device 86 includes a display 88 on which various screens may be displayed, including, in some embodiments, data from one or more of the screens discussed below. In some embodiments, caregiver assistance application 84 allows authorized users to remotely configure and remotely control various aspects of the patient support apparatuses 20 using their portable electronic device 86. Still further, caregiver assistance application 84 may be adapted to display information about the scale system of the patient support apparatuses 20, including any of the information discussed in greater detail below regarding the scale system.

In any of the embodiments disclosed herein, caregiver assistance application 84 may be configured to include any of the same features or functions as—and/or to operate in any of the same manners as—the caregiver assistance software applications described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. Thus, server 82 may be configured to share with one or more electronic devices 86 any of the information shared with the electronic devices disclosed in these aforementioned patent applications. For example, server 82 may be configured to not only share the location of patient support apparatuses 20 (and any devices that may be associated with them) with electronic devices 86, but it may also forward any of the data generated by patient support apparatuses 20 to the electronic devices 86, thereby letting the caregivers associated with these patient support apparatuses 20 know if, for example, the patient has exited patient support apparatus 20, what the patient's current weight is, whether the patient's cumulative weight change has exceeded a threshold or not, etc. Alternatively, or additionally, patient support apparatus server 82 may forward other patient support apparatus status data (e.g. current siderail position, bed exit status, brake status, height status, scale data, etc.) and/or caregiver rounding information (e.g. when the last rounding was performed for a particular patient, when the next rounds are due, etc.), and/or object data from any objects supported on patient support apparatus 20 to one or more electronic devices 86, thereby providing the caregivers associated with the devices 86 a consolidated portal (e.g. a single software application) for sharing this various information.

It will be understood that, although caregiver assistance application 84 is shown in FIG. 5 to be executed solely by server 82, this may be modified so that caregiver assistance application 84 is executed, either partially or wholly, on one or more of the mobile devices 86 as a mobile app that is downloaded to these devices 86. Similarly, caregiver assistance application 84 may be implemented as an app that executes on server 82 in conjunction with another specialized app that is downloaded to each of the mobile electronic devices 86. Still other variations of caregiver assistance application 84 are possible.

In some embodiments, patient support apparatus server 82 is also configured to determine the location of each patient support apparatus 20, or receive the location of each patient support apparatus 20 from the patient support apparatuses 20. In some embodiments, patient support apparatus server 82 determines the room number and/or bay area of each patient support apparatus 20 that is positioned within a room, as well as the location of patient support apparatuses 20 that are positioned outside of a room, such as, those that may be positioned in a hallway, a maintenance area, or some other area. In general, patient support apparatus server 82 may be configured to determine the position of any patient support apparatus 20 by communicating with one or more nearby wall units (not shown). Further details regarding several manners in which patient support apparatus 20 may be constructed in order to carry out such location communication, as well as the construction and/or operation of such wall units, are disclosed in the following commonly assigned U.S. patent applications: Ser. No. 63/245,245 filed Sep. 17, 2021, by inventors Kirby Niehouser et al., and entitled SYSTEM FOR LOCATING PATIENT SUPPORT APPARATUSES; Ser. No. 63/245,289 filed Sep. 17, 2021, by inventors Madhu Sandeep Thota et al. and entitled PATIENT SUPPORT APPARATUS COMMUNICATION AND LOCATION SYSTEM; Ser. No. 63/26,937 filed May 19, 2020, by inventors Alexander Bodurka et al. and entitled PATIENT SUPPORT APPARATUSES WITH HEADWALL COMMUNICATION; and Ser. No. 63/193,778 filed May 27, 2021, by inventors Krishna Bhimavarapu et al. and entitled PATIENT SUPPORT APPARATUS AND HEADWALL UNIT SYNCHING; the complete disclosures of all of which have already been incorporated herein by reference.

It will be understood that the architecture and content of local area network 78 will vary from healthcare facility to healthcare facility, and that FIG. 5 merely shows a generic example of the type of network a healthcare facility may be employ. Typically, one or more additional servers will be hosted on network 78 and one or more of them may be adapted to communicate with patient support apparatus server 82. For example, an electronic health record server will typically be present in any healthcare facility, and in some embodiments discussed herein, it will be in communication with patient support apparatus server 82 in order to receive patient data that is to be recorded in a patient's health record (e.g. weight readings taken from the scales built into patient support apparatuses 20; therapies provided to patients using a powered mattress onboard patient support apparatuses 20, etc.).

FIG. 6 illustrates a lock screen 90 that may be displayed on display 60 by controller 70 in response to one or more triggering conditions. In one embodiment, the triggering conditions include the initial power up of patient support apparatus 20, the passage of a predetermined amount of time during which control panel 56a is not used, and the user manually activating a control on control panel 56a that, when activated by the user, causes controller 70 to display the lock screen 90. In other embodiments, one or more of these triggering conditions may be omitted, supplemented, and/or replaced with other triggering conditions. In some embodiments, controller 70 is configured to turn off display 60 after a time period of disuse, and then, in response to a user touching anywhere on display 60, to display lock screen 90. Alternatively, controller 70 may be configured to display lock screen 90 before turning off display 60, or to not turn off display 60 at all. Controller 70 is therefore configured to display lock screen 90 after a predetermined amount of time passes without control panel 56a being used, regardless of whether it displays lock screen 90 immediately after the expiration of the predetermined time period, or only after the user presses somewhere on display 60. In some embodiments, the predetermined time period is about one minute, although other time periods can, of course, be used. In some embodiments, controller 70 is configured to allow the user to customize the predetermined time period.

The predetermined time period, in at least some embodiments, refers to the time period during which only control panel 56a is not used. In such embodiments, controller 70 is configured to still display lock screen 90 after the predetermined time period expires despite the fact that the patient may have used one of the inner siderail control panels 56c during that time period, and/or despite the fact that the caregiver may have used one of the outer siderail control panel 56b. In other embodiments, controller 70 may be configured to measure the predetermined time period with respect to both footboard control panel 56a and both outer siderail control panels 56b. In such embodiments, controller 70 is configured to display lock screen 90 only when all three control panels (56a, left control panel 56b, and right control panel 56b) are not used for the predetermined time period. In any of the embodiments herein, the non-use of a control panel 56 refers to the lack of touching anywhere on touchscreen display 60 as well as the lack of using any non-touchscreen controls 58 that are part of the control panel 56.

Lock screen 90 (FIG. 6) functions as a lock for using display 60. That is, controller 70 is configured to prevent a user from navigating to any other screens until the user activates an unlock control associated with lock screen 90. Thus, when controller 70 displays lock screen 90 on display 60, the user must activate the unlock control (described in greater detail below) before he or she is permitted to view and/or use any non-lock screens.

Lock screen 90 (FIG. 6) is configured in at least some embodiments to provide a tilt angle symbol 92 on lock screen 90 and a concealment control 58g. Tilt angle symbol 92, in the illustrated embodiment, is a generally rectangular structure positioned at the right edge of the upper corner of lock screen 90. Controller 70 is configured to change how it displays tilt angle symbol 92 in response to different values of the tilt angle—as detected by tilt sensor 74. For example, in one embodiment, controller 70 is configured to illustrate tilt angle symbol 92 in a first color when the tilt angle is zero degrees, and to illustrate the tilt angle symbol 92 in a second and different color when the tilt angle is non-zero. In some such embodiments, controller 70 displays tilt angle symbol 92 in a gray color when the tilt angle is zero and in a blue color when the tilt angle is non-zero.

Concealment control 58g is adapted to allow the user to selectively reveal and conceal the actual tilt angle measured by the tilt angle sensor 74. If a user presses on concealment control 58g in FIG. 6, controller 70 is configured to slide tilt angle symbol 92 in a leftward direction until it looks like what is shown in FIG. 7. Thus, FIG. 7 displays lock screen 90 when a user has utilized concealment control 58g to reveal the angle currently measured by tilt angle sensor 74.

As shown in FIG. 7, lock screen 90 additionally includes a tilt angle indicator 96 and a tilt icon 94 when the user has used the concealment control 58g to slide tilt angle symbol 92 leftward. Tilt angle indicator 96 shows the numeric value of the tilt angle being sensed by tilt angle sensor 74. Controller 70 is configured to update the numeric value of tilt angle indicator 96 in real time (or substantially real time, such as multiple times a second). Tilt icon 94 provides a graphic illustration of the manner in which litter frame 28 is currently tilted (or not tilted). In FIG. 7, tilt icon 94 has a horizontal orientation, which corresponds to the zero degree tilt shown by the tilt angle indicator 96 in FIG. 7. Tilt angle symbol 92 in FIG. 7 is displayed by controller 70 in the same color as it is displayed in FIG. 6 because the tilt angle is zero degrees for both of the situations illustrated in FIGS. 6 and 7.

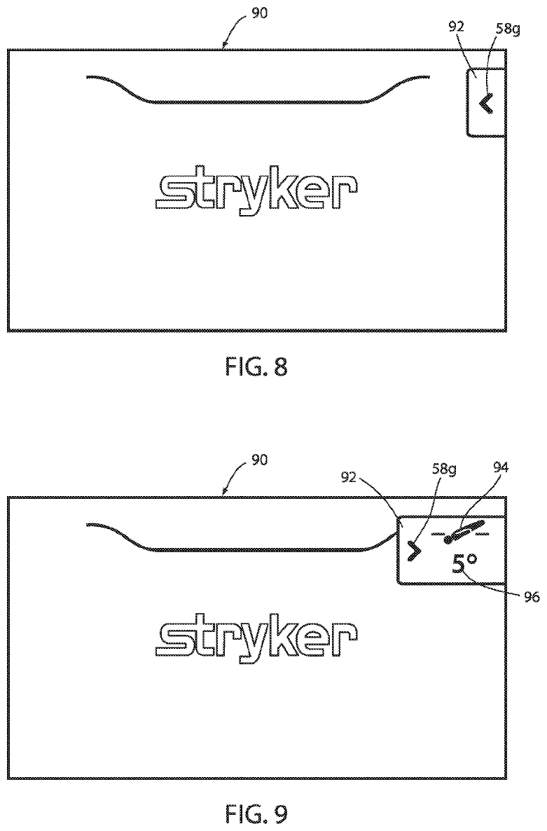

FIG. 8 shows an example of lock screen 90 when the current tilt angle of litter frame 28 is not zero and the concealment control 58g has been used to conceal the tilt angle indicator 96 (and tilt icon 94). As can be seen in FIG. 8, controller 70 is displaying tilt angle symbol 92 in a different color than what it displays tilt angle symbol 92 in FIG. 6. This is because the tilt angle of litter frame 28 in the example of FIG. 8 is non-zero. As was noted, in some embodiments, tilt angle symbol 92 is shown in blue when the tilt angle is not zero, although other colors may be used. Other characteristics of tilt angle symbol 92 may alternatively, or additionally, be changed when the tilt angle has a non-zero value, including, its size and/or shape.

If the user presses on the concealment control 58g shown in FIG. 8, controller 70 is configured to change the layout of lock screen 90 to what is shown in FIG. 9. That is, controller 70 slides the tilt angle symbol 92 leftward, revealing both the tilt icon 94 and the tilt angle indicator 96. As can be seen in FIG. 9, tilt angle indicator 96 shows that the current tilt angle is five degrees. Tilt icon 94 is changed (from what is shown in FIG. 7) to show the direction in which the litter frame is tilted. That is, tilt icon 94 includes a horizontal indicator and a patient indicator, and controller 70 is configured to change the orientation of the patient indicator in response to changes in the tilt angle. Thus, in the example of FIG. 8, the patient indicator has its head end lower than its foot end, thereby indicating that the litter frame 28 is in the Trendelenburg position in the example of FIG. 8.

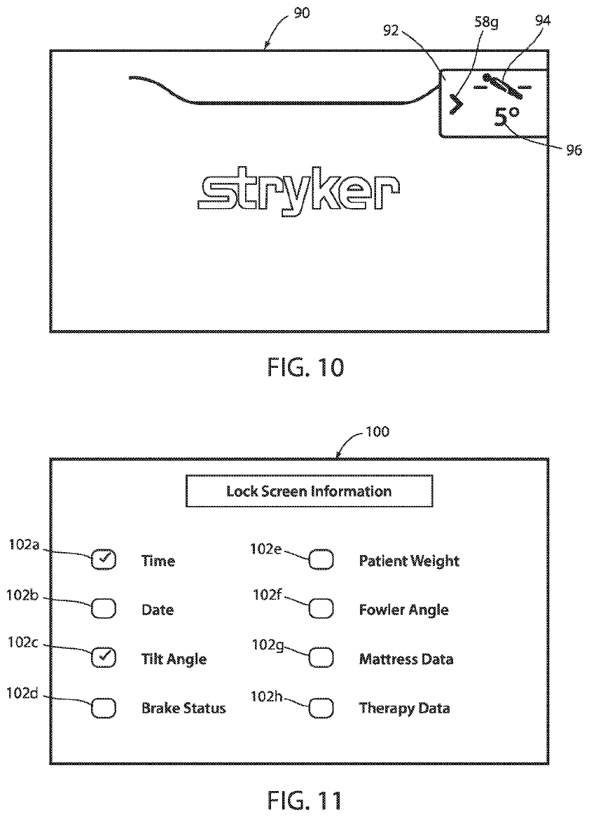

FIG. 10 illustrates the layout of lock screen 90 when the user presses on the concealment control 58g of FIG. 8 and the patient support apparatus 20 is tilted in a direction opposite to the situation of FIG. 9. That is, FIG. 10 illustrates lock screen 90 with an unconcealed tilt angle symbol 92 when the patient support apparatus 20 is in the reverse Trendelenburg position. As shown in FIG. 10, tilt angle indicator 96 shows that the current tilt angle is five degrees, and tilt icon 94 is displayed by controller 70 in a manner in which the patient indicator has its feet positioned lowered than its head (corresponding to the reverse Trendelenburg position). If the user wishes to conceal the tilt angle indicator 96 and tilt icon 94 shown in either FIG. 9 or 10, the user presses on concealment control 58g again, and controller 70 reverts to displaying lock screen 90 with the layout shown in FIG. 8. Concealment control 58g therefore acts as a toggle switch—alternating between concealing and revealing the tilt icon 94 and tilt angle indicator 96.

As shown in FIGS. 6-10, controller 70 is configured not to display any other information on lock screen 90 other than tilt angle symbol 92 and concealment control 58g (and, when unconcealed, tilt icon 94 and tilt angle indicator 96). In some embodiments of patient support apparatus 20, controller 70 is configured to allow a user to customize what information is shown on lock screen 90. For example, in some embodiments, controller 70 is configured to display a lock screen customization screen, such as customization screen 100 shown in FIG. 11. Lock screen customization screen 100 is displayed by controller 70 in response to a user using one or more controls 58 to navigate to it (and after the user has activated the unlock control of lock screen 90, discussed in greater detail below).

Lock screen customization screen 100 includes a plurality of check box 102a-h that the user may selectively check and uncheck. Whichever check boxes 102a-h that the user checks, controller 70 is configured to display the corresponding information on lock screen 90. Thus, in the example shown in FIG. 11, the time check box 102a and the tilt angle check box 102c are both checked. Controller 70 is therefore configured to display both the current time and tilt angle information (i.e. tilt angle symbol 92, concealment control 58g, and, when unconcealed, tilt icon 94 and tilt angle indicator 96) on lock screen 90. If the user were to select only check box 102f, controller 70 would display lock screen 90 with only information about the current angle of the Fowler section (e.g. the current angle from horizontal of head section 42). Customization screen 100 allows the user to select any one or more of the eight options shown thereon on lock screen 90. The user is also free to select none of the options shown in customization screen 100, in which case controller 70 displays no information on lock screen 90.

It will be understood that controller 70 may be configured to include a corresponding concealment control 58g for each of the options shown in customization screen 100. In other words, if the user selects the patient weight option by selecting check box 102e, controller 70 may be configured to display the patient's weight on lock screen 90 with a corresponding control 58g that, when activated, toggles between concealing and revealing the patient's currently measured weight. A similar corresponding concealment control 58g may be present for each of the other options shown in FIG. 11.

It will also be understood that the specific set of options shown in FIG. 11 may be varied in different embodiments of patient support apparatus 20. Thus, in some embodiments, controller 70 is configured to display more than the eight options shown in FIG. 11 on customization screen 100, while in other embodiments, controller 70 is configured to display less than these eight options on customization screen 100. Controller 70 may also be configured to provide the user with options that are different than the ones shown in FIG. 11. Still further, in some embodiments, controller 70 may be configured to provide the user with the ability to further customize the manner in which the selected data is displayed on lock screen 90 (e.g. the position of the displayed date, the size of the displayed data, the color of the displayed data, whether the displayed date has a concealment control or not, etc.).

In some embodiments, controller 70 is configured to allow the user to further customize the characteristic of symbol 92 that is changed in response to different tilt angles. For example, controller 70 is configured in some embodiments to allow the user to change one or more characteristics of symbol 92 based on the tilt angle being equal to (or not equal to) a particular non-zero angle, and/or the tilt angle being inside (or outside) of a particular range of angles. Thus, for example, in some embodiments, controller 70 might be customized by a user to display tilt angle symbol 92 in a first color when the tilt angle is zero, in a second color when the tilt angle is not zero but less than a user-customized value (e.g. five degrees), and in a third color when the tilt angle is equal to or greater than the user-customized value. Still other customizations of symbol 92 based on tilt angle values and/or ranges may be implemented by the user.

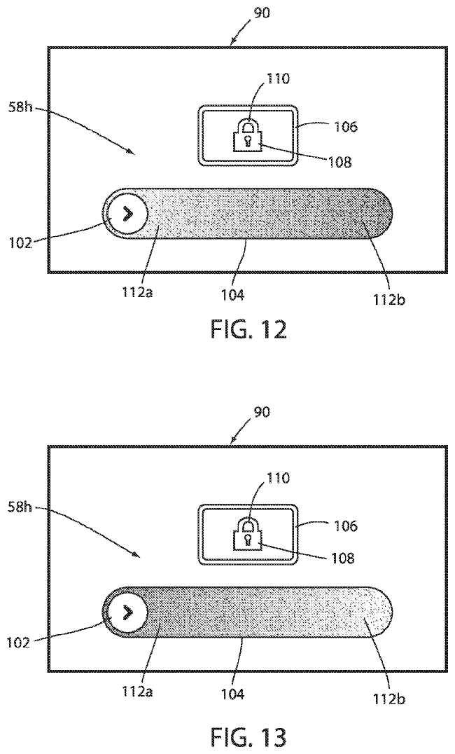

As was noted previously, controller 70 is configured to prevent the user from accessing any other screens while lock screen 90 is displayed. In order for the user to get past the lock screen 90 (and thus be able to see and/or to use those other screens), the user must first activate an unlock control 58h on patient support apparatus 20 (see FIGS. 12-19). In some embodiments, controller 70 is configured to display unlock control 58h for all the layouts of lock screen 90, while in other embodiments, controller 70 is configured to only display unlock control 58h after the user presses anywhere on lock screen 90 (other than one of the concealment controls 58g that may be present thereon). In the example illustrated in the accompanying drawings, controller 70 is configured to display lock screen 90 in the manner shown in FIGS. 6-10 (i.e. without unlock control 58h shown thereon) because the user has not pressed on lock screen 90 anywhere other than one of the concealment controls 58g. When the user touches any of the layouts of lock screen 90 shown in FIGS. 6-10, controller 70 is configured to stop displaying the information shown therein and switch to a layout of lock screen 90 that shows unlock control 58h, such as is shown in FIG. 12. In an alternative embodiment, controller 70 may be configured to add unlock control 58g in addition to what is shown in any of FIGS. 6-10 in response to a user touching lock screen 90. In still other embodiments, controller 70 may be configured to display unlock control 58h on lock screen 90 without requiring a user to first touch it.

As shown in FIG. 12, unlock control 58h includes a slider icon 102, a slider channel 104, a lock icon 106, a lock body 108, and a lock shackle 110. Slider channel 104 further includes a left end 112a and a right end 112b. As will be discussed in greater detail below, in order for a user to unlock the unlock control 58h, the user must press on slider icon 102 and drag it from left end 112a to right end 112b. Further, once slider icon 102 has been dragged to right end 112b, the user must continue hold their finger on slider icon 102 for a predetermined period of time. Thereafter, controller 70 switches to displaying a non-lock screen, such as, but not limited to, any of the motion control screens of FIGS. 20-24, a menu screen, an exit detection system control screen, a scale control screen, and/or another type of non-lock screen.

Prior to the user dragging slider icon 102 from the left end 112a of slider channel 104 to the right end 112b of slider channel 104, controller 70 is configured to provide a visual indication to the user of what movement is needed from the user in order to activate the unlock control 58h (i.e. unlock lock screen 90). In some embodiments, this visual indication is provided by controller 70 slightly changing the color of a portion of slider channel 104, wherein that portion starts at left end 112a and moves across slider channel 104 in a wavelike manner toward right end 112b (and then repeats). This wavelike motion moves in the same right-to-left fashion as the user must slide slider icon 102, thereby providing an indication to the user of the what direction he or she is to move slider icon 102.

FIGS. 12 and 13 illustrate this wavelike motion. In FIG. 12, left end 112a of slider channel 104 is a lighter shade of color than the rest of slider channel 104. This lighter shaded region of slider channel 104 is moved rightward by controller 70 until it reaches right end 112b of slider channel 104. FIG. 13 illustrates the situation where this lighter shaded region of slider channel 104 has reached right end 112b. After reaching right end 112b, controller 70 is configured to, in some embodiments, have the lighter shaded region continuing moving to the right until it disappears from slider channel 104, and then have it re-appear at left end 112a of slider channel 104.

In other embodiments, this wavelike motion may be indicated in other manners. For example, instead of a more lightly shaded region of slider channel 104 moving from left to right in slider channel 104, controller 70 could be configured to display a more darkly shaded region of slider channel 104 moving from left to right. As another alternative, controller 70 could be configured to have a region of slider channel 104 be displayed in a completely different color that then moves from left to right. As yet another alternative, controller 70 could animate an arrow moving from left to right, either in, or adjacent to, slider channel 104. In still other embodiments, controller 70 may be configured to display unlock control 58h without any animated movement indicative of the direction in which slider icon 102 is to be moved within channel 104.

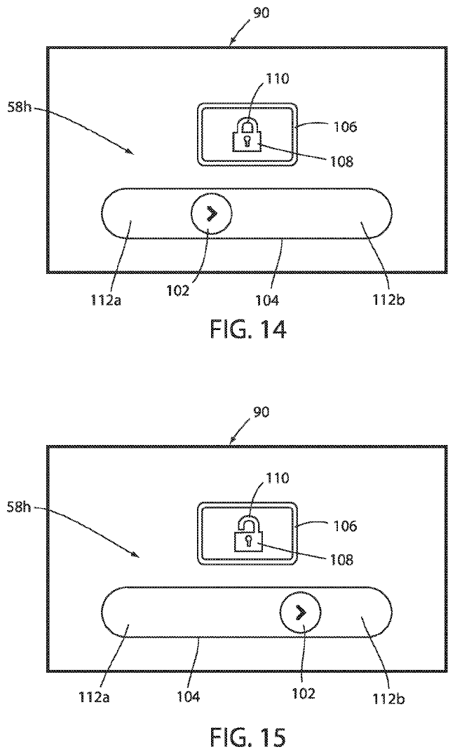

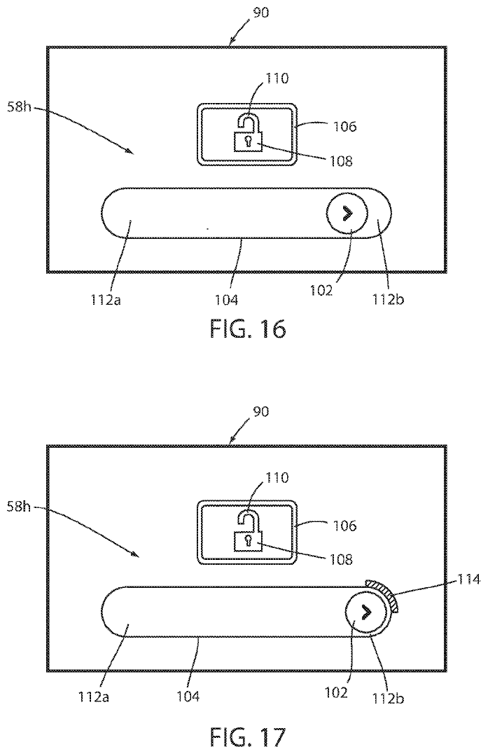

FIG. 14 illustrates unlock control 58h after the user has slid slider icon 102 a first portion of the distance from left to right in slider channel 104. FIG. 15 illustrates unlock control 58h after the user has slid slider icon 102 a second and greater portion of the distance from the left end 112a to the right end 112b of slider channel 104. FIG. 16 illustrates unlock control 58h after the user has slid slider icon 102 all of the way to the right end 112b of slider channel 104. As can be seen in these drawings (FIGS. 14-16), controller 70 is configured to animate lock icon 106 as slider icon 102 is moved from left to right.

Specifically, controller 70 is configured to animate lock icon 106 so that it appears to be unlocking as icon 102 moves from left to right. This animation can be seen in the changing height of lock shackle 110 with respect to lock body 108 in these drawings. In FIG. 14, where slider icon 102 has moved only a small portion of the way across slider channel 104, lock shackle 110 has moved vertically out of lock body 108 only a relatively small amount (compared to its position in FIG. 13). In FIG. 15, wherein slider icon 102 have moved further to the right, lock shackle 110 has moved vertically out of lock body 108 a greater amount. In FIG. 16, where slider icon 102 has moved even further to the right, lock shackle 110 has moved vertically almost to its full extent out of lock body 108. And finally, in FIG. 17, wherein slider icon 102 has moved all the way to the right, controller 70 has depicted lock shackle 110 as having moved vertically to its fullest extent out of lock body 108.

It will be understood that, although FIGS. 14-17 depict lock shackle 110 in four different discrete positions, controller 70 is configured to depict the movement of lock shackle 110 in a smooth and non-discrete manner. Further, the speed at which the lock shackle 110 is moved vertically upward by controller 70 is directly tied to the speed at which the user slides slider icon 102 across slider channel 104 from left to right. Thus, if a user quickly slides icon 102, lock shackle 110 will move vertically quickly; and if the user slides icon 102 slowly, lock shackle 110 will move vertically slowly.

As was noted previously, in order for the user to fully activate the unlock control 58h, the user must continue to press on slider icon 102 for a predetermined time period after it has reached right end 112b. In some embodiments, controller 70 is configured to draw a circle around slider icon 102 during this predetermined time period. In such embodiments, controller 70 is configured to draw this circle at a speed that is correlated to the predetermined time period so that, when the drawing of the circle is complete, the predetermined time period has expired.

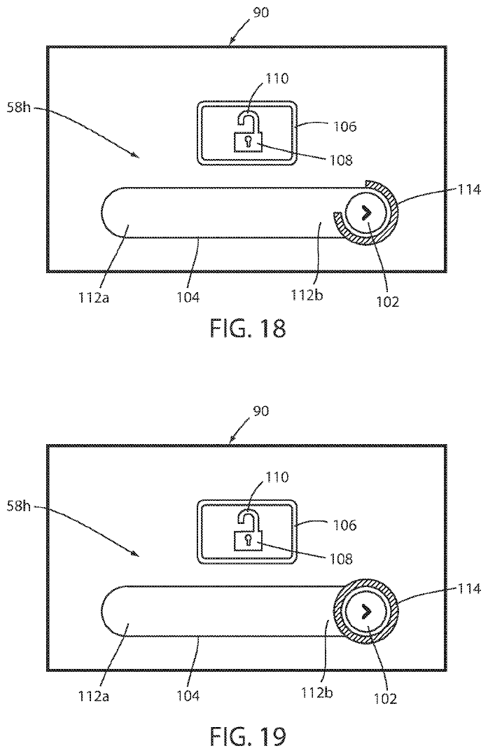

FIG. 17 illustrates a unlock control 58h after the user has continued to press on slider icon 102 for a first amount of time after slider icon 102 has reached right end 112b. As can be seen, controller 70 has only drawn a first portion of circle 114 at this moment. As the user continues to press his or her finger against slider icon 102, controller 70 continues to draw more and more of circle 114. FIG. 18 illustrates a moment in time subsequent to FIG. 18 after the user has continued to press slider icon 102 for a greater amount of time. As can be seen therein, controller 70 has drawn more of circle 114 around slider icon 102 in FIG. 18.

If the user continues to press his or her finger against slider icon 102, controller 70 eventually completes the drawing of circle 114. An example of this is shown in FIG. 19 where circle 114 is complete. Controller 70 displays FIG. 19 when the user has finished holding icon 102 for the entire predetermined time period. Controller 70 is then programmed to thereafter automatically display a different screen which, as noted above, may be any non-lock screen, such as, but not limited to, the motion control screens of FIGS. 20-24.

In some embodiments, if the user raises his or her finger off of slider icon 102 at any point during the unlocking process associated with unlock control 58h, controller 70 is configured to automatically move slider icon 102 back to left end 112a of slider channel 104. The user is thereafter required to retry the unlocking process by starting over with slider icon 102 at left end 112a. That is, the user must thereafter slide slider icon 102 all the way to the right end 112b of slider channel 104, and then keep his or her finger pressed on slider icon 102 for the predetermined time period. Any lifting of his/her finger during this process causes controller 70 to “rubber-band” the slider icon 102 back to its starting position at left end 112a of slider channel 104.

It will of course be understood that patient support apparatus 20 may be configured to implement a variety of different modification to the unlocking process of unlock control 58h. For example, in some modified embodiments, controller 70 may be configured to implement unlock control 58h without requiring the user to continue to press on slider icon 102 for a predetermined time period after sliding it to right end 112b of slider channel 104. In such modified embodiments, once the user has dragged slider icon 102 to right end 112b, controller 70 is configured to automatically switch to displaying a non-lock screen. In another alternative embodiment, controller 70 may be modified to eliminate the requirement of any sliding of icon 102, but instead only require the user to press and hold an icon for a predetermined period of time. In still other embodiments, controller 70 may be configured to implement unlocking control 58h by having the user slide slider icon 102 in a different direction, and/or in a plurality of directions. In still other embodiments, unlock control 58h may be implemented in still other manners.

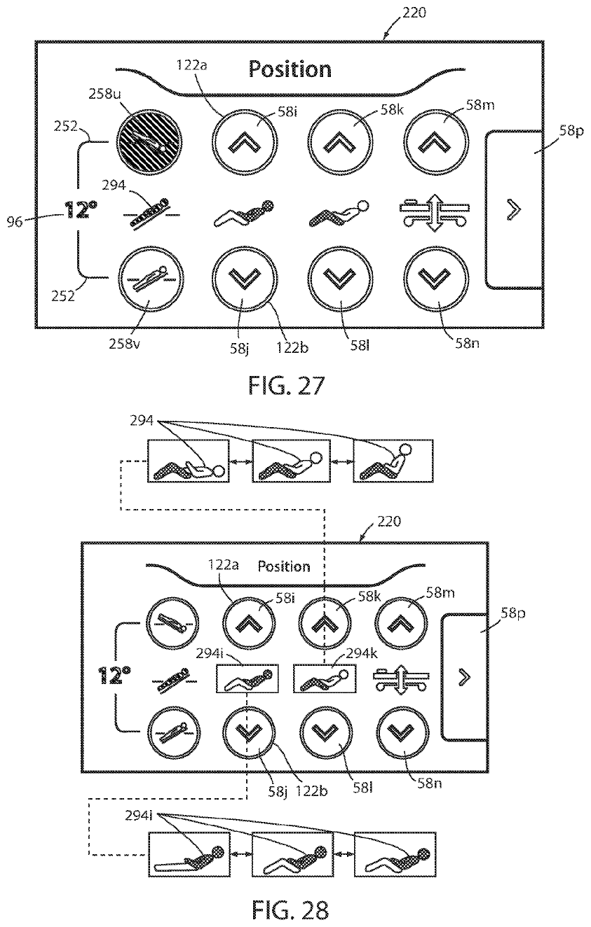

After a user has successfully unlocked the unlock control 58h, controller 70 is configured to display one or more non-lock screens on display 60 of control panel 56a. One of those screens is a motion control screen, such as the motion control screen 120 shown in FIGS. 20-24 or motion control screen 220 shown in FIGS. 26-28, 34, and 34A. Motion control screen 120 (or motion control screen 220) is also displayable in response to a user pressing on navigation control 58d (FIG. 4) of control panel 56a (provided the user has successfully unlocked the unlock control 58h). Motion control screen 120 (and motion control screen 220 include a plurality of motion controls adapted to allow the user to move various components of the patient support apparatus 20.

Motion control screen 120 (FIGS. 20-24) includes gatch lift and lower controls 58i and 58j, Fowler lift and lower controls 58k and 58l, litter frame lift and lower controls 58m and 58n, a CPR control 58o, and an arrow control 58p. When a user presses on Fowler lift control 58k, motion controller 64 sends a signal to activate Fowler actuator 66b (FIG. 5) causing it to raise head section 42. When a user presses on gatch lower control 58j, motion controller 64 sends a signal to activate Fowler actuator 66b in the opposite direction, causing it to lower head section 42. When a user presses on knee or gatch raise control 58i, motion controller 64 sends a signal to activate gatch actuator 66a causing it to raise the junction of thigh section 46 with foot section 48. When a user presses on knee lower control 58j, motion controller 64 sends a signal to activate gatch actuator 66a in the opposite direction, causing it to lower the junction of thigh section 46 and foot section 48. When a user presses on litter deck raise control 58m, motion controller 64 sends a signal to both head end lift 26a and foot end lift 26b, causing them to raise the overall height of litter frame 28. When a user presses on litter deck lower control 58n, motion controller 64 sends a signal to both head end lift 26a and foot end lift 26b, causing them to lower the overall height of litter frame 28.

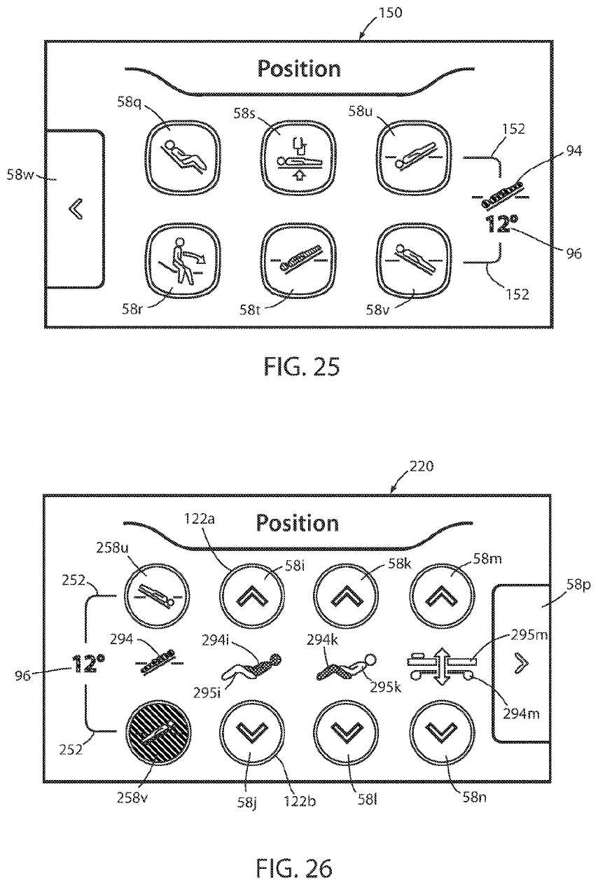

When a user presses on CPR control 58o (FIGS. 20-24), motion controller 64 sends signals to whichever ones of the actuators 26a-b and/or 66a-b that need to be moved in order to bring litter frame 28 and support deck 30 to a position suitable for performing CPR (e.g. litter frame 28 is level (and in some embodiments, at a specified height) and support deck 30 is flat). When a user presses on arrow control 58p, controller 70 is configured to display a supplemental motion control screen 150 (FIG. 25) that includes additional motion controls 58. These additional motion controls may include controls for moving patient support apparatus 20 to a plurality of predefined positions. In the example shown in FIG. 25, supplemental motion control screen 150 includes a chair position control 58q, an exit position control 58r, an examination position control 58s, a vascular position control 58t, a Trendelenburg position control 58u, and a reverse Trendelenburg position control 58v. Pressing on any of these controls causes controller 70 to activate the appropriate actuators 26a-b and/or 66a-b to move the patient support apparatus to the corresponding position. In the example shown in FIG. 25, supplemental motion control screen 150 also includes a pair of lines 152 that are directed from controls 58u and 58v, respectively, to a tilt icon 94 and a tilt angle indicator 96. Tilt icon 94 and tilt angle indicator 96 of FIG. 25 are the same as the tilt icon 94 and tilt angle indicator 96 of FIGS. 9-10. Tilt lines 152 may change color in the same manner as tilt icon 94 a tilt angle indicator 96. Supplemental motion control screen 150 also includes an arrow control 58w that, when pressed, causes controller 70 to return to displaying motion control screen 120 (FIGS. 20-24).

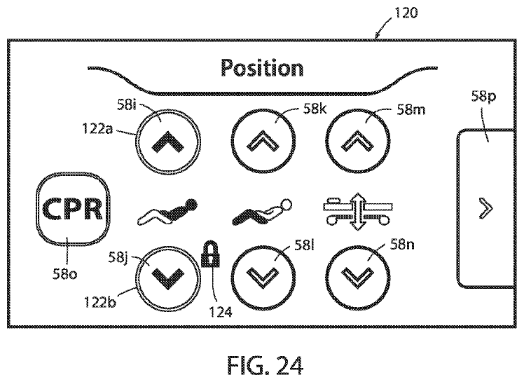

As was noted previously, control panel 56a (FIG. 4) includes a navigation control 58e that, when pressed, causes controller 70 to display a motion lock control screen (not shown). This motion lock screen allows a user to lock out any of the motion controls 58i-58n of FIGS. 24. When locked out, motion controller 64 does not respond to the pressing of any of the controls 58i-n, and the corresponding components of patient support apparatus 20 therefore do not move in response to the pressing of these controls. This motion lock out function is particularly useful in situations where a caregiver determines that a patient's position, orientation, and/or posture should not be disturbed. This motion lock out function applies to not only controls 58i-n of control panel 56a, but it also applies to the corresponding controls on control panels 56b and 56c. As a result, for example, if the gatch up/down controls 58i and 58j are locked, neither the patient nor the caregiver can use these controls on any control panels 56a-c to change the angle of Fowler section 42. In other embodiments, the lock out function only applies to selected control panels 56, such as the patient control panels 56c.

When a user has activated the lock out function of any of the motion controls 58i-n of motion control screen 120, controller 70 is configured to display a first lock indicator 122 on motion control screen 120. One example of this is shown in FIG. 21 wherein controller 70 has displayed first lock indicators 122a and 122b around the gatch motion controls 58i and 58h, respectively. First lock indicators 122a and 122b each comprise a circular shaped indicator that surrounds the circular perimeter of each of controls 58i and 58j. In some embodiments, each lock indicator 122a, b is yellow or amber colored, although other colors may be used. In general, each lock indicator 122a, b is of a color that contrasts with the color of the corresponding control 58 (e.g. controls 58i and 58j in FIG. 21).

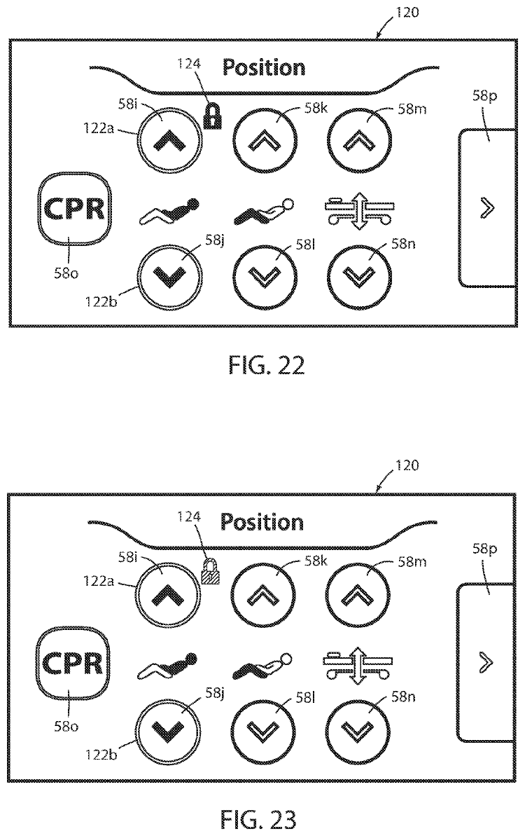

Controller 70 is configured to display a first lock indicator 122 at all times on motion control screen 120 for each of the controls 58i-j that are currently locked out. Controller 70 is also adapted to selectively display a second lock indicator 124 on motion control screen 120 when a user presses on a particular control 58i-j that is currently locked out. One example of this is shown in FIG. 22. In FIG. 22, controller 70 has added a second lock indicator 124 positioned adjacent gatch raise control 58i, which is currently locked out. Controller 70 adds the second lock indictor 124 to motion control screen 120 when the user actually presses on the locked out control. The second lock indicator 124 is the same color as first lock indicator 122, in some embodiments. In other embodiments, indicators 122 and 124 may have different colors. In general, both indicators 122 and 124 should have colors that contrast well with the surrounding color so that they can be more easily seen.

Controller 70 is configured to stop displaying second lock indicator 124 after the user stops pressing on the locked out control 58. In some embodiments, controller 70 is configured to display the second lock indicator 124 as gradually fading away after the user stops pressing on the corresponding locked out control 58. One example of this is shown in FIG. 23 where second lock out indicator 124 is displayed in a faded form. FIG. 23 therefore illustrates motion control screen 120 a brief moment after the user has stopped pressing on gatch raise control 58i. The amount of time that controller takes to fade away the second lock indicator 124 may vary from embodiment to embodiment. In some embodiments, it may be on the order of 1-2 seconds. As noted, controller 70 is configured to not fade away first lock indicators 122, regardless of whether a user is pressing on the corresponding control 58 or not. Thus, in the example of FIG. 23, controller 70 will continue to display first lock indicator 122 at its normal brightness for as long as motion control screen 120 is displayed (and the gatch controls 58i and 58j remained locked out).

Controller 70 is configured to display second lock indicator 124 adjacent whichever locked out control 58 the user presses on. Thus, the position of second lock indicator 124 will change for different controls 58. FIG. 24 provides one illustration of this. In the situation shown in FIG. 24, a user has pressed on Fowler lower control 58j, which is locked, and controller 70 has displayed second lock indicator 124 adjacent Fowler lower control 58j.

The use of both first and second lock indicators 122 and 124 provides the user with a better understanding of the motion control lock out function. The first indicator 122 lets the user know that one or more motion controls are locked out, and is displayed at all times that motion control screen 120 is displayed. If the user forgets the significance of first indicator 122, or otherwise does not notice it, controller 70 is configured to add second lock indicator 124 to the screen 120. The addition of second lock indicator 124 to the screen 120 in response to a user pressing on a locked out motion control 58 lets the user know that controller 70 has sensed the user pressing on the control, but that motion will not take place because that particular control is locked out. This selective display of the second lock indicator 124 therefore lets the user know that the control panel 56 is still operational, while re-emphasizing that motion will not take place because of the current locked out status of the corresponding motion control 58.

Motion control screen 220 (FIGS. 26-28) is similar to motion control screen 120 and includes gatch lift and lower controls 58i and 58j, Fowler lift and lower controls 58k and 58l, litter frame lift and lower controls 58m and 58n, and an arrow control 58p, which allows a user to switch between control screens. Thus, for example, when a user presses on or touches gatch lift control 58i, motion controller 64 sends a signal to activate the gatch actuator causing it to move the gatch section 42. For further details of the signals and the controller's corresponding activations in response to input by a caregiver using the various controls 58i, 58j; 58k, 58l; and 58m, 58n, reference is made to the above description. For further details of gatch lift and lower controls 58i and 58j, Fowler lift and lower controls 58k and 58l, litter frame lift and lower controls 58m and 58n, and arrow control 58p reference is also made to the above description.

Referring again to FIG. 26, in addition, motion control screen 220 includes Trendelenburg position controls 258u, 258v—one to tilt the deck of the patient support into a Trendelenburg position (“Trend”) and the other to tilt the deck of the patient support into a reverse Trendelenburg position (“reverse Trend”), similar to controls 58u and 58v. In addition, similar to motion control screen 120, motion control screen 220 includes an icon 294 with each pair of controls (e.g. 58i, 58j; 58k, 58l; 58m, 58n; and 258u, 258v). As will be more fully described below, each icon may be a dynamic icon and configured to provide a visual representation of the component being controlled by the control similar to icon 94.

For example, the icon 294 for the Trendelenburg position may be a graphical representation of the deck arranged in a flat configuration with a person lying in a supine position on the graphical representation of the deck and located and vertically aligned between controls 258u and 258v. Further, icon 294 may include a reference line, such as horizontal line. Optionally, as noted, icon 294 may be a dynamic icon that moves, for example by tilting, when the Trendelenburg control or the reverse Trendelenburg control is touched or pressed to provide graphical visual feedback to the caregiver who is adjusting the litter orientation. For example, when the deck is flat and the litter is horizontal (for example when the litter angle is 0+/−0.5 degrees)—the icon 294 will be shown in a horizontal orientation (and aligned with the horizontal line in the icon). When the deck is in a Trend position, where for example the litter angle is greater than 0.5 degrees, the icon will be shown with the head end of the deck and the person's head tilted below the horizontal line. When the deck is in a “Reverse Trend” configuration, where for example the angle is greater than 0.5 degrees, the icon will be shown with the foot end of the deck and the person's feet tilted below the horizontal line. The icon, therefore, may just have three orientations—horizontal, Trend, or Reverse Trend. Optionally, the icon may also be moved to show incremental angles of the litter—such as 5 degrees, 10 degrees, 15 degrees etc.

Located next to icon 294 is a display of the litter angle 96 of the expressed in a numerical form, similar to as shown in FIG. 25, except in this instance it is located to the left of, but aligned with icon 294. Similarly, motion control screen 220 may also include lines 252 (similar t lines 152) to visually tie the litter angle to the Trendelenburg controls 258u, 258v so that user will understand that the angle represents the Trend angle. Further, lines 252 may have a contrasting color (to the background design, which is shown as dark grey) and optionally may change color to further convey what Trend position is being adjusted using the controls. Lines 252 may have the same color as the controls 258u, 258v. For example, as shown, when a user presses or touches or presses control 258v, the control background will change color and optionally to the same color as icon 294, for example blue as shown in FIG. 26. The angle 96 may also be displayed or shown in the same color as the icon 294 when the respective control is activated. Thus, motion control screen 220 may use a unified color scheme to indicate when the Trend controls are being operated and which one is being activated and to what angle the deck is being oriented. This angular feedback information may be limited and provided in a discrete number of angles—for example, for three preselected angles—or may cover the full range of angles and adjusted in small increments—for example, such as in one degree or five degree increments.

Similar to motion control screen 120, motion control screen 220 may also include icon 294i for the gatch controls (aligned vertically between the gatch controls), which is a graphical representation of a person in a tilted seated position where the gatch section of the deck is raised. Further the portion 295i of the icon 294i that corresponds to the person's body (i.e. legs) supported by the gatch section of the deck (“gatch corresponding portion of the icon”) may be illustrated in one color while the remaining portion of the image that corresponds to the remaining portion of the person's body is illustrated in a second color. For example, the gatch corresponding portion 295i of the icon 294i may be white, while the other or remaining portion may be grey to provide a clear contrast between the portions of the icon and to indicate which portion is being controlled by the respective control.

Optionally, as best seen in FIG. 28, when the gatch section of the deck is moved, for example, using controls 58i or 58j, icon 294i may be dynamic. For example, controller 70 may change icon 294i to reflect the angle of the gatch section, similar to icons 94 and 294 described above. In this manner, the dynamic icon may provide a graphical visual feedback of the gatch angle to the caregiver who is adjusting the gatch section of the deck. For example, the gatch corresponding portion 295i of the icon may move to show the angular orientation of the gatch section of the deck as controlled by the respective gatch control, such as shown in FIG. 28.

Referring again to FIGS. 26, 27, and 28, motion control screen 220 may also include icon 294k for the Fowler controls (aligned vertically between the Fowler controls), which may also be a graphical representation of a person in a tilted seated position where the Fowler section of the deck is raised. Similarly, the portion 295k of the icon 294k that corresponds to the person's body (i.e. back) (“Fowler corresponding portion of the icon”) supported by the Fowler section of the deck may be shown in one color while the portion of the image that corresponds to the remaining (e.g. non-moving) portion of the person's body may be shown in a second color. For example, the Fowler corresponding portion 295k of the icon may be white, while the other or remaining portion may be grey, again to provide a contrast between the two portions of the icon—corresponding to the portion of the deck that is being moved and to the other portion that is not.

Optionally, when the Fowler section of the deck is moved, for example, using controls 58k or 58l, icon 294k may also be dynamic and change to reflect the angle of the Fowler section, similar to icon 294i. For example, the Fowler corresponding portion 295k of the icon 294k may move to show the angular orientation of the Fowler section of the deck (as controlled by control 258k) and, therefore, also provide a graphical visual feedback of the Fowler angle to the caregiver who is adjusting the Fowler section of the deck, such as shown in FIG. 28.

Similarly, motion control screen 220 may have an icon 294m that is a graphical representation of the patient support, illustrating the deck and the base, with the movement (up or down) of the deck (and hence extension or contraction of the lift assembly) represented by a double headed arrow 295m to convey that the controls 58m, 58n can raise or lower the deck similar to motion control screen 120. Arrow 295m may have the same color as the deck and base portion of the icon or may have a different or contrasting color. Icon 294m may also be dynamic and show a separation between the deck and the base of the icon increasing when the deck is raised and reducing when the deck is lowered. Thus, icon 294m can also provide user feedback to the caregiver of the caregiver's adjustment to the deck or litter height.

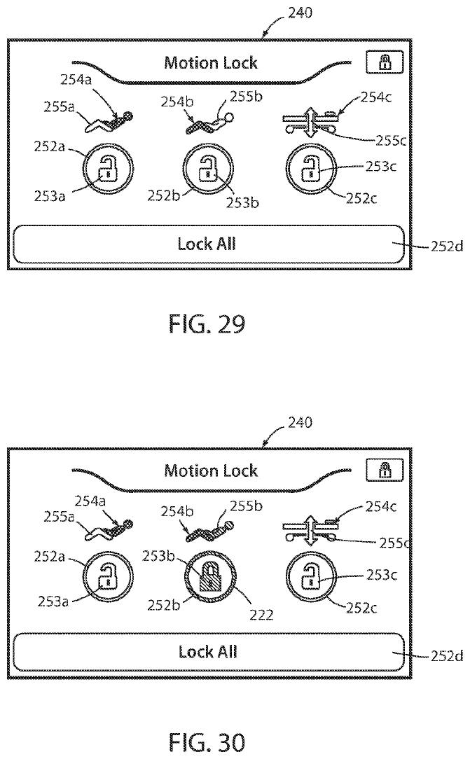

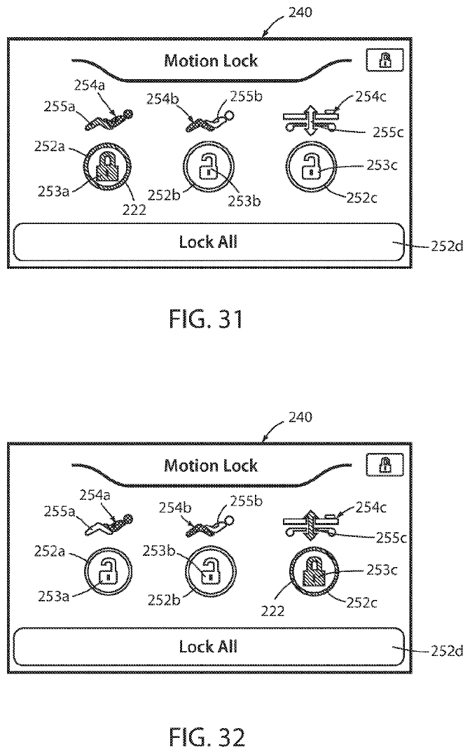

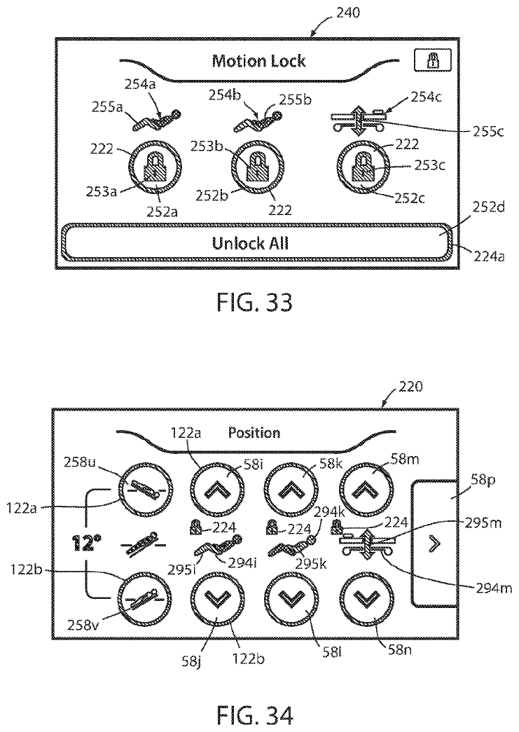

As was described previously, control panel 56a (FIG. 4) includes a navigation control 58e that, when pressed, causes controller 70 to display a motion lock control screen. Referring to FIGS. 29-33, motion lock screen 240 allows a user to lock out any of the motion controls 58i-58n, 258u, and 258v of FIGS. 26-28. When locked out, motion controller 64 does not respond to the pressing or touching of any of the controls 58i-n, 258u, or 258v, and the corresponding components of patient support apparatus 20, therefore, do not move in response to the pressing or touching of these controls.

As best seen in FIGS. 29-33, motion locking screen 240 includes a plurality of lock controls 252a, 252b, 252c. Each lock control is associated with a component of the patient support that can be locked. For example, lock control 252a is associated with the gatch section of the deck of the patient support. Lock control 252b is associated with the Fowler section of the deck of the patient support. Lock control 252c is associated with the height of the deck to raise or lower the deck relative to the patient support base and/or casters. Optionally, motion locking screen 240 includes an all lock or unlock control 252d that can be used to lock or unlock all the components (or a group pf components), such as those noted above with a single control. When the control 252d is pressed or touched, it may provide an indication that it is in a locked mode or state. For example, similar to controls 58i-58n, lock control 252d may have a lock indicator in the form of a perimeter outline 224a of a contrasting color, such as yellow or amber.

Each control may have an icon 253a, 253b, and 253c in the form of a graphical representation of a lock and, further, which is a dynamic icon that changes, either its color and/or its configuration to indicate a locked mode or state. Thus, icons 253a, 253b, and 253c form lock indicators similar to lock indicators 124 described above.

As shown in FIGS. 29-33, when unlocked, the icons 253a, 253b, and 253c may comprise a graphical representation of a lock in an unlocked configuration (e.g. shackle is shown opened) and change to a graphical representation of a lock in a locked configuration (e.g. shackle closed and locked) and optionally in another color. Therefore, instead of only appearing when the lock control is touched or pressed, they may be displayed at all times and, instead, change their configuration and/or color when the lock control is activated.

Additionally, as shown in FIGS. 30-33, similar to controls 58i-58n described above in reference to motion control screen 120 and below in reference to motion control screen 220, motion lock screen 240 may have lock indicators 222 in the form or annular rings that extend around each of the controls, which change color when the controls are in a locked state. Further, the color of the dynamic icon within the control may correspond to the color of the indicators to provide a unified color scheme indicating that the control is in a locked state.

Associated with each individual lock control (252a, 252b, and 252c) is another icon 254a, 254b, 254c. Icons 254a, 254b, 254c may be a graphical representation of the component of the patient support that is being locked. For example, similar to icon 294i, icon 254a is a graphical representation of a person in a tilted seated position where the gatch section of the deck is raised and lockable, and with the portion 255a of the icon that represents the portion of the person supported by the gatch portion shown in a contrasting color, for example, white. Similar to icon 294k, icon 254b is also a graphical representation of a person shown in a tilted seated position where the Fowler section of the deck is raised and lockable, and with the portion 255b of the icon that represents the portion of the person supported by the Fowler portion shown in a contrasting color, for example, white. Similar to icon 294m, icon 254c is a graphical representation of the patient support, illustrating the deck and the base, with the movement (up or down) deck represented by a double headed arrow 255c conveying that the deck can be raised or lowered and can be locked. Arrow 255c may have the same color as the deck and base portions of the icon or may have a different or contrasting color. As noted, icons 254a-c may be similar to icons 294i, 294k, and 294m, again to provide a consistent and easily understood user interface that makes it easy for a caregiver to understand the status of the patient support at a glance, accommodating the variations in caregivers preferences for sign posting and agnostic to a caregiver's language.

One or more, or all, of icons 254a, 254b, and 254c may change color or have portions that change color when they are in a locked state to indicate they are in a locked state. For example, the portions 255a, 255b of icons 254a and 254b shown in FIG. 29 may change to the same color as the lock controls and lock indicators-such as yellow or amber. Similarly, the portion 255c of icon 254c representing the movement of the deck, in this case the arrow, may also change color. Thus, when activated in a locked state or mode, portions 255a, 255b, and 255c all may change to the same color as the lock control and the lock indicator, for example yellow or amber. In this manner, motion lock screen 240 can provide a unified color scheme (i.e. a single common color) associated with the control(s), lock indicator(s), and icon(s) to indicate a locked state of one or more components.

Similar to icons 294i, 294k, and 294m, and 294, icons 254a, 254b, and 254c may have portions that move when the angle or height of the component has been previously changed using the control icons of motion control screen of FIG. 26-28. For examples of how thee icons can change reference is made to the above description of icons 294i, 294k, 294m, and 294 and FIG. 28.

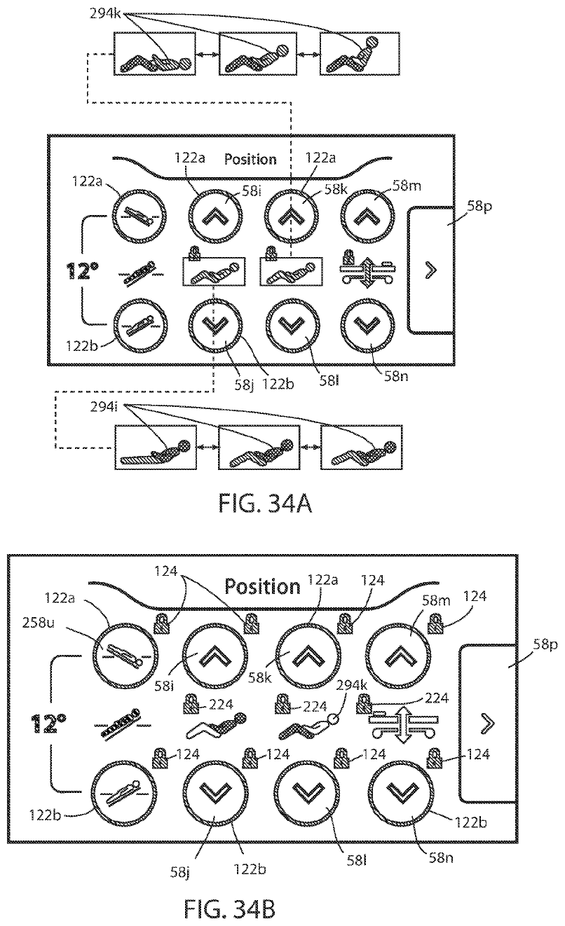

When one or more controls of the patient support components are locked, the controls and associated icons on the motion control screen 220 may change similar to motion control screen 120. As best seen and understood from FIG. 34, when the gatch section, the Fowler section and/or the vertical movement of the deck is locked, the corresponding controls 58i and 58j; 58k and 58m; 58m and 58n, will be greyed out to indicate that the control is no longer operable to control the respective component. In addition, as noted above, lock indicators 122a and 122b may be displayed. Further, if a caregiver presses or touches any one of the locked controls, another lock indicator will appear 124 while the control is being pressed or touched. For further details of lock indicators 122a, 122b and 124, reference is made above to motion control screen 120.

Thus, when a user has activated the lock out function of any of the motion controls 58i-n of motion control screen 220, controller 70 is similarly configured to display first lock indicators 122a and 122b on motion control screen 220 and lock indicators 124 when a user touches or presses a locked out control. Thus, the lock out indicators are responsive to a selection made by a user and let the user know that the controls will not allow adjustment to the deck configuration or height of the deck. As will be more fully described, certain selected functions may override the locked status, for example, the CPR control and/or the vascular position control 258u, described below.

As best understood from FIGS. 34, 34A, and 34B, motion control screen 220 also may include a third lock out indicator 224, similar to lock out indictor 124, which are displayed in response to a user pressing on a locked out motion control to let the user know that controller 70 has sensed the user pressing on the locked out motion control, but that motion will not take place because that particular control is locked out. While all the lock indicators 124 are displayed on the screen shown in FIG. 34B (to show their respective locations adjacent the motion controls), controller 70 is configured to stop displaying second lock indicator 124 after the user stops pressing on the locked out control. For further optional details of lock out indicator 124, reference is made to the above description.

Lock out indicator 224 may similarly comprise an icon in the image of a lock (illustrated with its shackle in a locked configuration). Lockout indicators 224 may be located adjacent each of icons 294i, 294k, and 294m and are shown after the respective motion controls have been locked out using lock control screen 240 and remain there until the respective motion control is unlocked. Therefore, lock out indicators 224 may be static--meaning they keep their configuration and color until no longer displayed.

Similar to indicators 122, 124, indicator 224 may be colored, such as yellow or amber colored, to match the color of indicators 122a, 122b and/or 124 to provide uniform color scheme or coding that is in contrast to the respective control. It should be understood that other colors may be used. But in general, each lock indicator 122, 124, and 224 is of a color that is in contrast with the color of the corresponding control 58 (e.g. controls 58i and 58j shown in FIGS. 34, 34A, and 34B).

To further or alternately provide feedback to the use to the state of the controls (e.g. locked on unlocked), icons 294i, 294k, and 294m may also be color coded and, optionally, match the color of the lock indicators to provide user feedback that a particular portion of the patient support is locked. For example, the Fowler corresponding portion 295k of the icon 294k may have the same color as the lock indicators 122a, 122b, such as yellow or amber, when the Fowler section of the deck is locked. Similarly, the gatch corresponding portion 295i of the icon 294i may have the same color as the lock indicators, such as yellow or amber, when the gatch section of the deck is locked. Arrow 295m of icon 294m may also have the same color as the lock indicators, such as yellow or amber, when the lifting and lowering function of the deck is locked to provide a uniform color scheme or coding and quick reference to or reinforcement of the status of the particular component of the patient support 20.

When a portion of the deck or the height of the deck is locked, controller 70 may generate the lock out indicator 224 adjacent the respective icon 294i, 294k, and/or 294m, while locked. Therefore, lock out indicator 224 together with first and second lock indicators 122 and 124 provide the user with a better understanding of the status of the motion control lock out function.

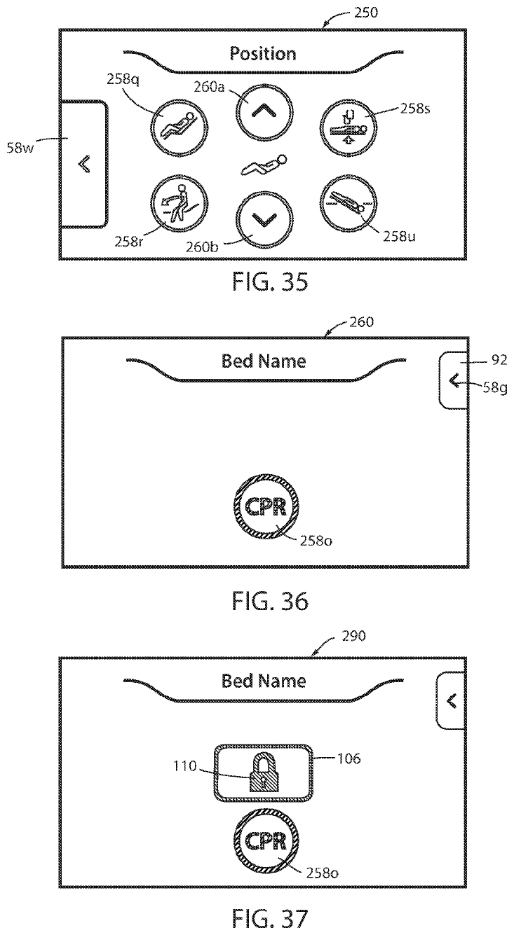

When a user presses on arrow control 58p, controller 70 may be configured to display an alternate supplemental motion control screen 250 (FIG. 35) that includes additional motion controls similar to supplemental motion control screen 150. For example, these additional motion controls may include controls for moving patient support apparatus 20 to a plurality of pre-set or predefined positions. In the example shown in FIG. 35, supplemental motion control screen 250 includes a chair position control 258q, an exit position control 258r, an examination position control 258s, and a vascular position control 258u, similar to controls 58q, 58r, 58s, and 58u described above. Pressing on any of these controls causes controller 70 to activate the appropriate actuators to move the deck and/or litter to the corresponding position. When the individual controls, e.g. fowler control, gatch control, or lift and lower controls, are locked, each pre-set configuration control may be also locked out (and also color coded to indicate they are locked out with color rings similar to the controls 122a, 122b described above), though, as noted above, one or more pre-set controls, such as pre-set controls associated with safety issues, such as the CPR control, a Fowler control that provides an angle pre-set of 30 degrees or greater than 30 degrees, or a vascular position control, may be configured to override the locked state.

In addition, supplemental motion control screen 250 includes auto contour motion controls 260a, 260b for adjusting the gatch and Fowler sections at the same time. Optionally each of controls 258q, 258r, 258s, and 258u has an icon associated therewith to indicate the function of the respective control. Controls 260a, 260b on the other hand have icons in the form of arrows to indicate that the angles of gatch/Fowler sections are to be increased or decreased—simultaneously. Additionally, an icon 260c may be provided between controls 260a, 260b that is a graphical representation of a person in a tilted seated position, which also may be dynamic, similar to icons 294i, 294k and 258a, and 258b, described above. Further, icon 260c is located between controls 258q, 258s and between 258r, 258u. Controls 258q, 258r, 258s, and 258u and 260a, 260b and icon 260c may also be color coded to indicate when any of these pre-set conditions or when the gatch or Fowler are locked out. Typically, most, if not all, of these pre-set conditions have clinical relevance and are provided to allow a caregiver easy and quick control of the deck and/or litter to achieve the pre-set configuration without requiring individual control over each actuator, and further as noted may be configured to override a locked state of the respective actuator(s).

Thus, controller 70 may be configured adapted to provide a uniform color coding of one or more of the motion controls and the lock controls when one or more of the motion controls is locked by one or more of the lock controls. Similarly, in addition or alternately, controller 70 may be configured to provide a uniform color coding of the icons associated with the motion controls and the lock controls when one or more of the motion controls is locked by one or more of the lock controls. Further, the uniform color coding may be supplemented by providing various lock indicators that may also be color coded and in some cases dynamic to provide additional feedback.

Referring to FIG. 36, rather than locating the CPR control 590 (FIG. 24) on the motion control screen, controller 70 may display a CPR control on a home display screen 290 so that before any control screens are displayed the CPR control will be displayed for easy access by the caregiver. Similar to screen 90, when the patient support is locked out, screen 290 may have a lock icon 106 with a graphical representation of a lock with its lock shackle 110 in a locked configuration, which indicates no controls over the patient support are available, though optionally one or more controls associated with safety or clinical relevance may be configured to override the locked state of the relevant actuator.

In some embodiments, controller 70 is configured to transmit data regarding the locked status of one or more controls 58 to patient support apparatus server 82 via transceiver 68 (FIG. 5). In such embodiments, patient support apparatus server 82 and/or caregiver assistance application 84 may be configured to forward this information to one or more electronic devices 86. These devices may then display this information on display 88 of the corresponding device. In this manner, remotely positioned caregivers can be apprised of which controls on the patient support apparatus 20 are currently locked out, and which ones are currently not locked out.

In some embodiments, the mobile electronic device 86 and/or caregiver assistance application 84 are adapted to allow the mobile electronic device 86 to send commands to the patient support apparatus 20 to remotely control one or more of the aspects of the patient support apparatus 20. For example, in some embodiments, mobile electronic device 86 is adapted to allow a user to remotely move components of patient support apparatus 20, and may be configured to display a motion control screen on display 88 that is similar to, or the same as, the motion control screen 120 of FIGS. 20-24 or motion control screen 220 of FIGS. 26-28, 34, and 34A.

It will be understood that the lock screen and motion lockout concepts disclosed herein may all be combined together in a patient support apparatus 20, or they may be individually integrated into a patient support apparatus 20. For example, in some embodiments, patient support apparatus 20 includes all the features discussed herein relating to the lock screen 90, but does not include any of the lock out indicators 122 and 124 of motion control screen 120 (or motion control screen 220). Alternatively, in some embodiments, patient support apparatus 20 includes one or more of the lockout indicators 122 and 124 on motion control screen 120 or 220, but does not include any or all of the features of the unlock control 58h of lock screen 90 (or doesn't include a lock out screen at all). In still other embodiments, still other combinations of the screens and functions disclosed herein may be integrated into a patient support apparatus 20.

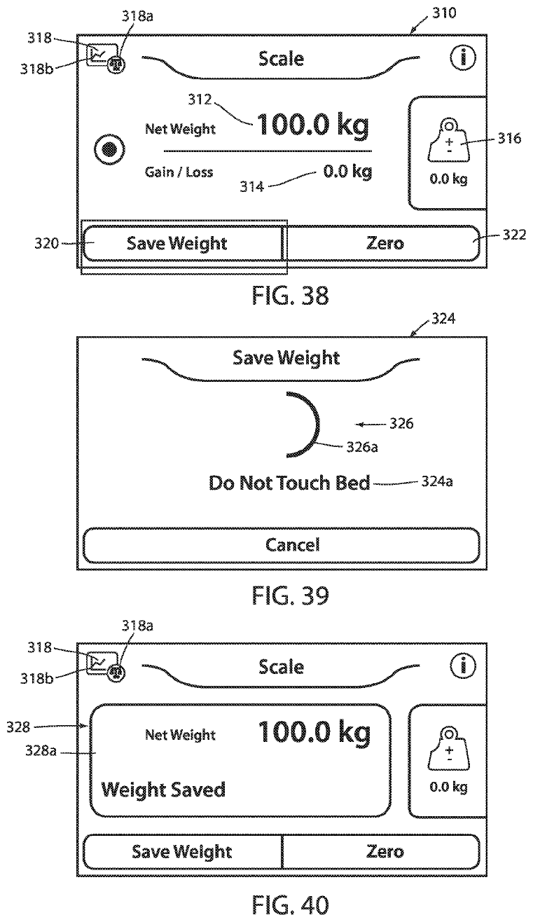

As was described previously, control panel 56a (FIG. 4) includes a navigation control 58c that when selected, e.g. when pressed or touched, by the user generates a signal to cause controller 70 to display a scale or weighing control screen, such as scale or weighing control screen 310. Referring to FIGS. 38-40, scale control screen 310 may include a weight display area 312 to display the current weight of a patient (as measured by the apparatus scale or weighing system) and a gain/loss display area 314 to display the change in weight of the patient as compared to previous day. Alternately or in addition the gain/loss may display the change as compared to the initial weight and, therefore, convey the total weight loss or gain. Additionally, control screen 310 may include a unit control 316 that when selected (e.g. pressed or touched) by a user allows the user to change the units of measurement for the displayed weight of the patient—e.g. from lb to kg or vice versa. Control screen 310 may also include a weight history control 318 that when selected by the user generates a signal to cause the controller to display the weight history of the patient, as measured by the weighing process of the weighing system and saved in memory at the patient support apparatus, as will be more full described below.

Further, control screen 310 may include an input control 320 that when selected by the user generates a signal to cause the controller to save the current weight of the patient in the local patient records of the memory on the patient support apparatus and optionally may be transmitted to a remote memory, such as in the EMR. In addition, control screen 310 may include a zero control 322 that when selected by the user generates a signal to cause the controller to zero out the scale of the patient support apparatus, for example, before a patient is supported on the patient support apparatus.

When a user presses or touches input control 320, control 320 generates a signal to cause the controller 70 to save the current weight of the patient, which process is not instantaneous and requires several readings over a weighing time period and hence takes time to save the weight. Therefore, while controller 70 is saving the patient's weight, controller 70 generates a save weight display screen 324 over the scale control screen 310 (it can cover the whole scale control screen or just cover part of it). Save weight display screen 324 displays instructions 324a, for example, words such as “DO NOT TOUCH BED” or a symbol (e.g. hand in a circle with a line through it) so a caregiver understands not to touch the patient support apparatus during the saving process, which could interfere with the accurate weighing of the patient.

In addition, save weight display screen 324 may also have a progress indicator 326 so that a caregiver understands the progress of the saving weight process (e.g. how much time has passed out of the total time in the saving weight process). For example, the progress indicator 326 may comprise numerical values (e.g. a percentage) or a dynamic icon that changes to covey how much progress the controller has made in the saving weight process. In the illustrated embodiment, the progress indicator is a dynamic icon in the form of a ring or an annular line 326a in a contrasting color (to the background of display 324) that starts out with the ring or annular line only partially shown and then progresses to fill in until the ring or annular line is filled in 360 degrees once the saving process is complete. The progress indicator may take other forms as noted including a pie shaped icon that fills in (starting with a small slice and then fills into until the whole pie shaped icon is a solid contrasting color) or a bar that is partially filled in with a contrasting color and then fully filled in once the saving process is complete, or an arrow that points to a scale on a line. Additionally, the indicators may have marks that provide additional detail on the progress, for example, short bars or lines at 25%, 50%, 75%, and then 100%. In each case, the indicator fills in or moves as the weighing process progresses to provide the user with feedback on where the controller is in the weight saving the process. This feedback allows the caregiver to assess how long they will need to not touch the patient support apparatus and, optionally, allow them to draw their attention to other activities knowing how long their attention can be directed elsewhere.

Once the progress indicator shows the weight saving process is complete, the controller may return scale control display screen 310 but also to then display an indication that the weight was saved. For example, controller may generate a box 328 around the current weight value, optionally in a contrasting color, and a further indication 328a, such as the words “WEIGHT SAVED.” After a period of time, such as a few seconds, controller 70 will return to scale control screen 310. Optionally, the controller may generator another completed process indicator-including a sound or flashing display to let the caregiver know the weight saving process is complete in addition indicator 328.

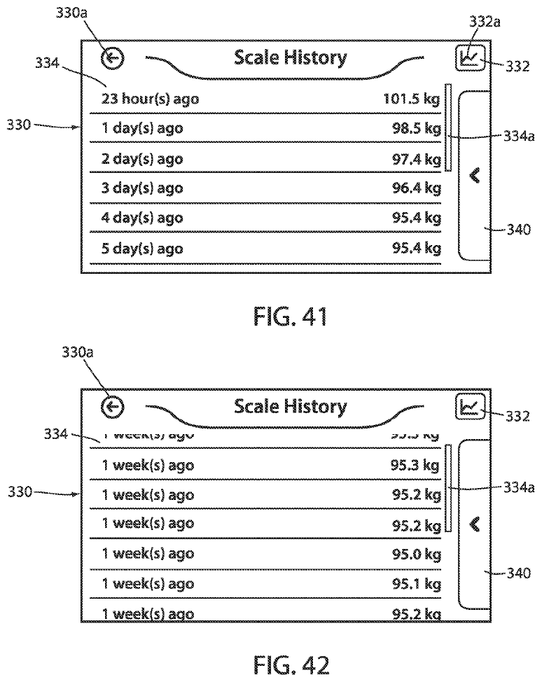

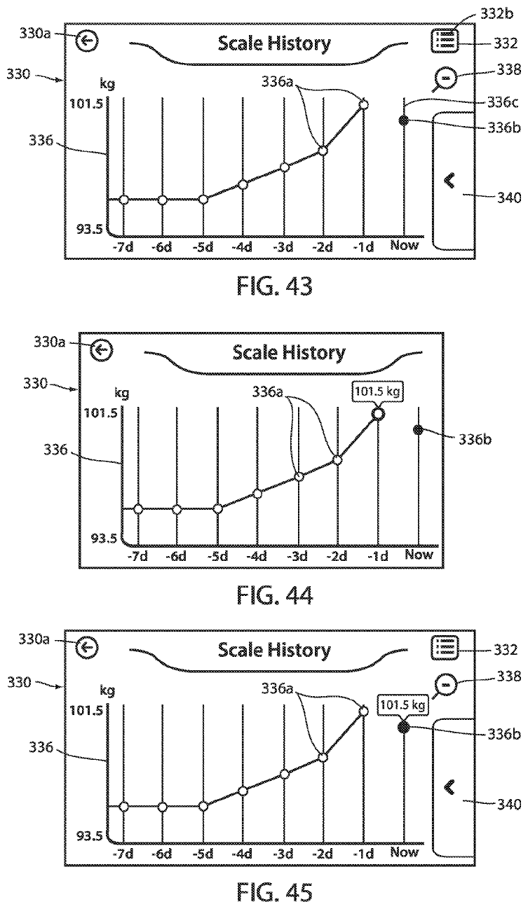

Referring to FIGS. 41-42, as described above, scale control screen 310 may include a history control 318 that when selected by the user generates a signal to controller 70 to generate a history display screen 330 at the display 60. History control 318 may include one or more icons associated with the function of history control 318, such as icon 318a, which indicates the function of the history control 318, for example, a graphical representation of a scale, and icon 318b, which indicates a format associated with the history control 318, such as a graph format (see FIGS. 38 and 40) or a list format (see e.g. FIG. FIG. 43).

As best seen in FIG. 41, display screen 330 includes a return control 330a that when selected generates a signal to controller 70 to return to the scale control display screen 310. History display screen 330 is configured to provide the user with a history of the patient's weight (as measured by the weighing system and saved in memory) and, further, to provide a format control 332 to allow a user to select the format for the history. For example, display screen 330 may be configured to display the patient's weight history data in one format, such as a listing or table 334, including a partial listing or table, or in another or second format, such as a chart (see FIGS. 43-48 described below). Optionally, format control 332 may have an icon 332a that is a graphical representation of one format, for example, a graph, and a second icon 332b (FIG. 43), which is a graphical representation of one format, for example, a listing. The format control icon will switch to the icon representing the format not being displayed-that is when the format control with the graph format is selected, and the graph format is displayed on history display screen 330, the icon will switch to the (listing) icon 332b. When the format control with the listing format is selected, and the listing format is then displayed, the icon will switch to the (graph) icon 332a.

Depending the length of the history, controller 70 may configured the listing or chart to display a period or a subset of the patient's weight history, and optionally is configured to allow a user to scroll up or down the entire listing, for example, either by dragging the listing or chart. For example when in the listing format, controller may generate a scroll bar 334a to the side of the listing to allow a user to scroll up or down beyond the displayed period. The period may be adjustable. For example, the controller 70 may configured the listing or chart to display a week of the patient's weight history, and optionally may generate a settings control to allow a user to adjust or select the period of time for display or the length of time period between each data point. In one aspect, the settings may allow the entire history to be displayed or controller 70 may generate a zoom control to allow the user to adjust the period as discussed below. In another aspect, as noted below, if the graph is configured to display just 1 day, then the settings will also allow the user to select how often the weight is measured, e.g. every hour or couple of hours.

When display screen 330 is in the listing format and the format control 332 is selected by a user, the control will generate a signal to controller 70 to switch or toggle to a chart format of the patient's weight data, for example, a graph 336 (see FIGS. 43-48). Graph 336 may be a two-dimensional graph and have a horizontal orientation with the time period on the X-axis of the graph and the weight on the Y-axis, or it may be reversed to have a vertical orientation with the time period on the Y-axis and weight on the X-axis. Similarly, when display screen 330 is in the chart format and the format control 332 is selected by a user, it will generate a signal to controller to switch or toggle to the listing or table 334 of the patient's weight data. Optionally format control 332, as noted, may include a dynamic icon that changes to indicate the alternate format-e.g. icon 332a, which is a representation of a chart when display 330 is in the listing format and icon 332b, which is a representation of a listing when display 330 is in the chart format.

Similar to listing or table 334, graph 336 may display the patient's weight of each predetermined time period (e.g. each day) and, optionally, include user interfaces 336a for each data point for each time period (e.g. each day) so a user can select, by pressing or touching, the user interface 336a to get an exact weight in numerical format (see e.g. FIG. 44) for that time period (e.g. day) versus trying determine it from the weight axis scale. When a user selects a data point, the user interface 336a may change format and/or color, as described below, to indicate which data point is selected.

As best seen in FIG. 45, chart 336 may also include a separate line 336c and separate weight data point for the current day and current weight, which includes a user interface 336b that when selected shows the current weight in a numerical format (FIG. 45). The user interface 336b moves along line 336c as the users weigh changes during the day. This allows a user to quick determine the current weight of the patient and can quickly compare to the previous weight of the patient with a quick glance at the line. Once a 24 hour period is passed, that data point then is added to the adjacent graph and a new data point will be generated for the new day on line 336c.

User interfaces 336a and 336b may be circular icons and, further, dynamic, as noted, in that they change color and/or have a contrasting ring that appears around the circular icon when it is selected so that it is clear to the user which data point they are selecting (FIG. 44).

As noted above, the time period that is covered by the displayed history may be preselected—e.g. a default time period of a week. This time period may be adjusted, either by changing the default, as noted above, in the settings, or by a chart size control 338. When the number of days included in the display screen is enlarged (as described below, see e.g. FIG. 47), this feature of the user interfaces changing color and/or changing shape (e.g., adding the annular ring in a contrasting color) may facilitate user feedback on which data point the user is selecting. Optionally, if the time period is too large to show each day on the time axis, the corresponding day for the selected data point may appear on the time axis (e.g. pop up) when a data point is selected between the illustrated time periods (as shown in FIG. 47 fewer time periods may be displayed on the time axis to avoid overcrowding of the data).

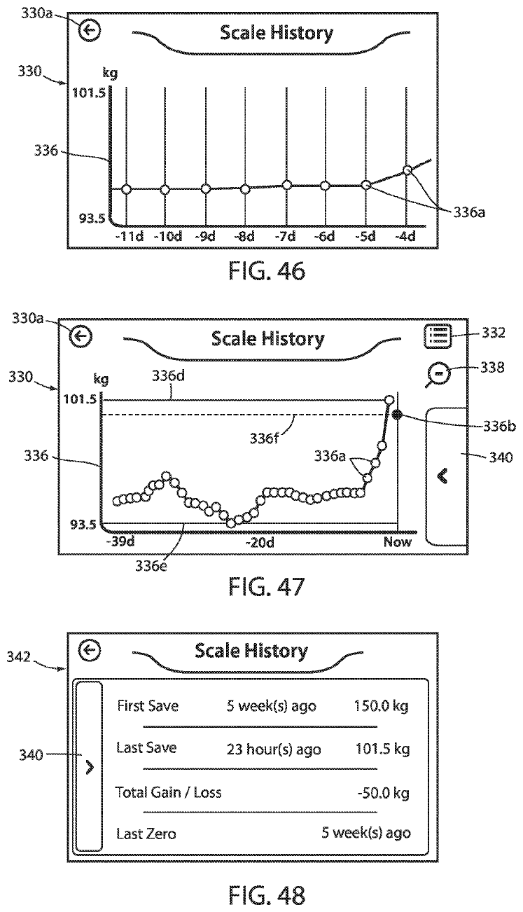

As best understood from FIGS. 46-47, similar to listing 334, chart 336 may include daily weight history that starts with the first day the patient was supported on patient support apparatus (see FIG. 47) up to and including the current weight, as well the day on which the weight data corresponds, e.g. 1 day ago, 2 days ago. Similar to listing 334, chart 336 may be configured to allow a user to scroll along the time axis (e.g. by dragging the chart) to see the older data and, further, may be enlarged or reduced in size (zoomed in or zoomed out) using a chart size control 338. When selected by the user, input at chart size control 338 causes the controller to reduce the displayed data (hence make the chart data less compact) or expand the displayed data (and hence make the chart data more compact) to allow less data or more data, including all the data, to appear on the display screen, as shown in FIG. 47. For example, the user may just want to look at less than 7 days (including one day where the time axis is in hours instead and the single vertical current weight is updated every hour) or more than a week up to and including the entire history. As would be understood, the fidelity or granularity of the data may be reduced when the data is expanded to include the patient's first day, especially when the patient has been on the patient support apparatus for a considerable period of time, hence the ability to distinguish a selected data point from other data points may be very helpful.

To help put the patient's weight in perspective, the chart may have references lines. For example, referring to FIG. 47, graph 336 may include reference lines 336d, 336e, and/or 336f. Line 336d may be a line (e.g., horizontal line when the horizontal axis is the weight axis) that represents the maximum weight of the patient, line 336e (e.g., horizontal line when the horizontal axis is the weight axis), and line 336f (e.g., horizontal line when the horizontal axis is the weight axis) may represent the current weight of the patient. These lines may appear, as shown, on the full weight history display or may also, or instead, be displayed on the default period version of the weigh history (e.g. FIGS. 43-46) or an adjusted period version. Further, the lines may be coded using color or dashes to distinguish between the lines. For example, the max and min weight lines 336d and 336e may be solid lines in one color (but contrasting to the axis lines), while the current weight line 336f may be dashed as shown in a contrasting color to the other two lines, as well as to the axis lines. These lines may be static and appear the whole time or may be dynamic, e.g. flash or change color, to draw the attention of the caregiver when certain parameters are met (e.g. weight loss or weight gain exceeds a preselected or a selectable percentage) or may appear only for a period of time.

Display 330 (in both listing and chart formats) may include another format control 340 to allow a user to switch between a full weight history or a condensed weight history. As best seen in FIG. 48, when control 340 is selected by the user, control 340 generates a signal to controller 70 to toggle or slide between a condensed history display screen 342 and the full weight history display screen (see FIGS. 43-48 and 41-42). Condensed history display screen 342 may display a reduced set of weight information, such as the weight of the patient when it was first saved and also the time period that corresponds to the first saved weight (“the first saved weight date”). Condensed history display screen 342 may also display the last saved weight and the time period that corresponds the last saved weight and display the total weight gain or loss. Optionally, condensed history display screen 342 may also display the last date when the apparatus was zeroed, which may or may not be the same as the first saved weight date.

In this manner, when a user is viewing the weight history, the user may quickly switch or toggle between a full weight history and a condensed history at the display by simply selecting control 340, which may remain in the same location the screen or switch sides of the screen as shown.

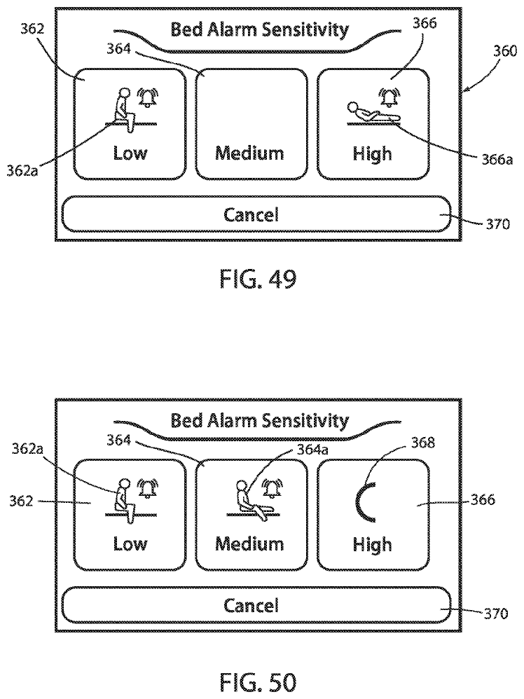

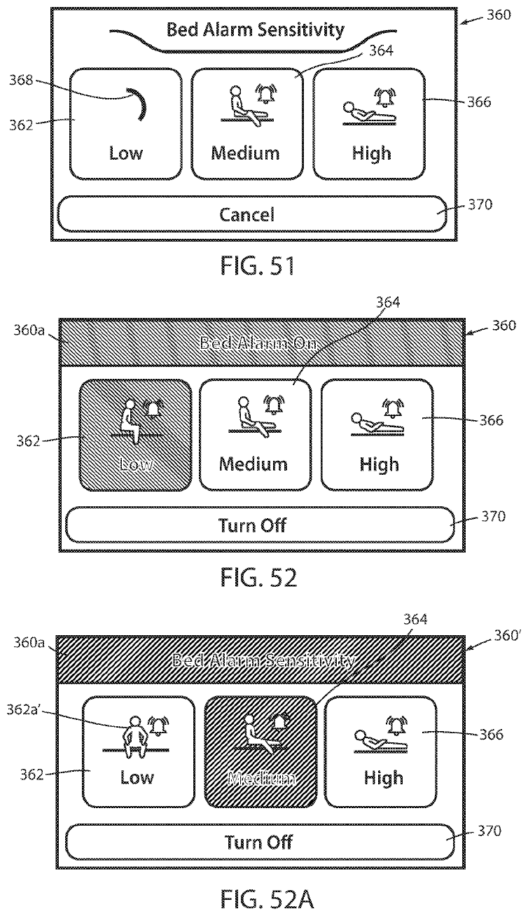

As described above, when a user presses navigation control 58a, control panel 56a may display an exit detection control screen on display 60 that includes one or more icons that, when touched, control an onboard exit detection function. Referring to FIGS. 49-52, when a user selects navigation control 58a, exit detection control screen 360 may be displayed on display 60. As will be more fully described below, exit detection control screen 360 is configured to allow a user to select between different sensitivity levels or settings for the exit detection system and, further, switch between sensitivity levels before the exit detection system is “fully” set or armed for a selected sensitivity level. For example, this may be useful when a caregiver initially selects one sensitivity level but then realizes that the patient is not properly positioned on the patient support apparatus for the selected sensitivity level, and rather than wait for the exit detection system to go through arming process steps with the wrong sensitivity level, the controller can instead immediately switch the sensitivity level to the second (or third) selected sensitivity level on the exit detection system.

Referring again to FIGS. 49-52, the exit detection system includes exit detection control screen 360 generated by controller 70 at display 60. Exit detection control display screen 360 may be configured with two or more sensitivity level controls to allow a user to select between two of more different sensitivity settings. For example, detection control display screen 360 may be configured with three sensitivity level controls 362, 364, and 366 to allow a user to select between three different sensitivity settings, for example, (1) a low sensitivity level that a user may select at control 362 so that the exit detection screen only issues an alarm when a patient is exiting the patient support apparatus; (2) a medium sensitivity level that a user may select at control 364 so that the exit detection screen only issues an alarm when a patient is nearing exiting the patient support apparatus; and (3) a high sensitivity level that a user may select at control 366 so that the exit detection screen issues an alarm when a patient is moving sufficiently, for example, to indicate the patient is awake. Control display screen 360 also includes a control 370 to allow a user to cancel or turn off the exit detection system (cancel when the arming process in still in progress or turn off when the arming process is complete or “armed”).

Optionally each control 362, 364, and 366 may have a graphical icon 362a, 364a, and 366a, respectively, associated with the control, which is a graphical depiction of a person supported on the patient support surface (e.g. mattress) in a configuration that suggests the patient's movement associated with the sensitivity level. For example, icon 362a is a graphical depiction of person seated and leaning forward with both the legs over the side of the apparatus to indicate the patient is exiting the bed. Icon 364a is a graphical depiction of person seated with one leg over the side of the apparatus to indicate the patient is nearing exiting the bed. Icon 366a is a graphical depiction of person with their back slightly raised but both legs on the patient support apparatus to indicate the patient is awake.

Once a control (362, 364 or 366) is selected, controller 70 communicates the selected sensitivity level to the exit detection system, which then initiates the arming process of the patient support apparatus based on the selected sensitivity level. The arming process occurs or extends over a period of time, for example, a few seconds depending how quickly the loads cells can get an accurate reading and the desired accuracy. As would be understood when the weight readings are used for dosing a patent, the load cells' accuracy is important. Therefore, the time period might be slightly longer in those circumstances.

In addition, controller 70 replaces the graphical depiction of the person on the selected control with a progress indicator 368, for example, an annular line or ring that fills in to indicate the progress of the arming process of the exit detection system similar to progress indicator 326. Once fully armed, the controller 70 again shows the graphical depiction of the person (see FIG. 52) (associated with that control) but with a contrasting color background from the previously unselected state of the control (e.g. see FIGS. 49-51). In addition, the controller 70 displays a notification 360a in the banner that the exit detection system is armed, for example, using the words “BED ALARM ON”, optionally with the same contrasting color background used on the selected control. Notification 360a may be located over the exit detection control display screen label, namely “BED ALARM SENSIVITITY” and remains displayed until the exit detection system is turned off using control 370.

As noted described above, controller 70 is configured to allow a user to switch the sensitivity level of the exit detection system before the system is armed even after making an initial selection. In other words, a caregiver can select (using controls 362, 364, or 366) a first selected sensitivity level for the exit detection system. Before the exit detection system is fully armed (before the arming time period has finished) for the first selected sensitivity level, e.g. also before it indicates the “BED ALARM ON” status shown in FIG. 52, the controller is configured to allow the user to select another sensitivity level, e.g. a second selected sensitivity level, using another of the controls (362, 364, or 366), which will generate a signal to the controller to interrupt the arming process (based on the first selected sensitivity level) so that controller 70 will change the sensitivity to the second selected sensitivity level selection (or third selected sensitivity level if another change is needed) made by the user before the arming of the exit detection system based on the first selection is complete.

Optionally, as shown in FIG. 52A, the label in the banner in exit detection control screen 360′, namely “BED ALARM SENSIVITITY”, may remain displayed even when the alarm is on and, further, may use color to indicate or provide notification 360a that the alarm is on. For example, the banner may have a background with a color associated with an alarm (e.g. red) to indicate that the alarm is on rather than or in addition to the words “BED ALARM ON”, which optionally matches the background color (e.g. red) on the control of the selected sensitivity. Further, exit detection control screen 360′ may incorporate a modified icon 362a′ for control 362, which is a graphical representation of a person facing (outwardly as shown in FIG. 52A) the edge of the support surface (mattress) with their legs over the edge of the support surface and with their arms resting on their legs—which may be an even clearer illustration of a person exiting the patient support apparatus. For further details of control screen 360′, reference is made to control screen 360 described above.

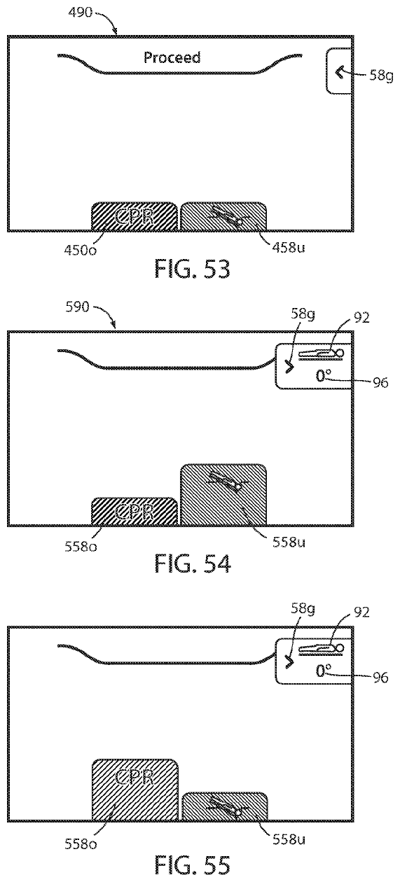

Referring to FIG. 53, the numeral 490 generally designates another home screen. Home screen 490, which is similar to above described home screens, includes a CPR button 458o and vascular button 458u to provide immediate access to the CPR and vascular controls in addition to concealment control 58g. In the illustrated embodiment, buttons 458o and 458u are configured as tabs and further are color coded in contrasting colors—with the CPR tab being colored red, while the vascular tab is colored on amber. As will be more fully described below the buttons may be configured to be dynamic, for example, change in size, color and/or have a dynamic icon associated with the tab to further provide feedback to the caregiver.

Referring to FIGS. 54 and 55, the numeral 590 generally designates another home screen. Home screen 590, which is similar to above described home screens, includes a CPR button 558o and vascular button 558u to provide immediate access to the CPR and vascular controls in addition to concealment control 58g. In the illustrated embodiment, buttons 558o and 558u are also configured by the controller GUI as color coded tabs but are dynamic buttons or tabs in that they change size when selected. For example, when selected both tabs, which are rectangular in shape, increase in size along their respective vertical axes (as viewed from a caregiver) to provide a larger touch area.

In addition, the controller may be configured to automatically open the tilt angle symbol 92 to display the current angle 96 of the deck when either of the CPR button or vascular button is selected.

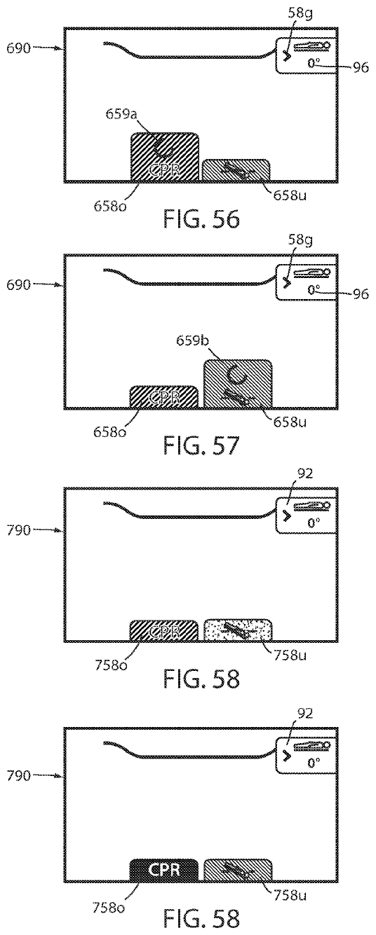

Referring to FIGS. 56 and 57, the numeral 690 generally designates another home screen. Home screen 690, which is similar to above described home screens, also includes a CPR button 658o and vascular button 658u to provide immediate access to the CPR and vascular controls in addition to concealment control 58g. In the illustrated embodiment, buttons 658o and 658u are also configured by the controller GUI as color coded tabs and are also dynamic buttons or tabs in that in addition to changing size when selected. For example, when selected both tabs, which are rectangular in shape, increase in size they each incorporate an annular spinner 659a, 659b that indicates motion is in progress for the selected CPR or vascular function.

Referring to FIGS. 58 and 59, the numeral 790 generally designates another home screen. Home screen 790, which is also similar to above described home screens, includes a CPR button 758o and vascular button 758u to provide immediate access to the CPR and vascular controls in addition to concealment control 58g. In the illustrated embodiment, buttons 758o and 758u are also configured by the controller GUI as color coded tabs and are also dynamic buttons or tabs in that in addition to changing color when selected. For example, when CPR button 758o is selected it changes from red to purple. Vascular button 758u chances from amber to light maroon. It should be understood that the colors noted are exemplary only.

The controller may also be configured to automatically open the tilt angle symbol 92 on home screen 790 when either of the CPR button or vascular button is selected so that the current angle 96 of the deck is displayed.

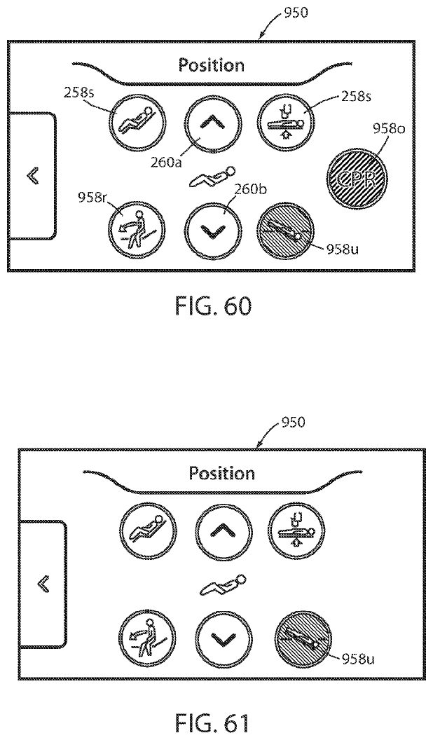

Referring to FIGS. 60 and 61, the numeral 950 generally designates a supplemental motion control screen, which is similar to supplemental motion control screen 250 and includes controls for moving patient support apparatus 20 to a plurality of pre-set or predefined positions. In the example shown in FIG. 60, supplemental motion control screen 950 includes a chair position control 258q, an exit position control 258r, an examination position control 258s, and a vascular position control 958u, similar to controls 58q, 58r, 58s, and 58u described above. In addition, as best seen in FIG. 60, supplemental motion control screen 950 may also includes CPR button 958o. Pressing on any of these controls or buttons causes controller 70 to activate the appropriate actuators to move the deck and/or litter to the corresponding position. When the individual controls, e.g. fowler control, gatch control, or lift and lower controls, are locked, each pre-set configuration control may be also locked out (and also color coded to indicate they are locked out with color rings similar to the controls 122a, 122b described above), though, as noted above, the pre-set controls associated with safety issues, such as the CPR control button 958o and vascular position control button 958u may be configured to override the locked state.

To enhance the user feedback and flag those pre-set positions that might be more urgent, vascular button 958u and the optional CPR button 958o and are color coded—red for CPR and amber for vascular position function.

For further details of the supplemental motion control screen 950 reference is made above to the description of supplemental motion control screen 250.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient thereon;
an actuator adapted to move a component of the patient support apparatus;
a control panel including a first motion control;
a display; and
a controller adapted to switch the first motion control between an active state and a locked state, wherein when the first motion control is in the active state, the controller is adapted to activate the actuator in response to a user pressing on the first motion control, and wherein when the actuator is in the locked state, the controller is adapted to not activate the actuator in response to the user pressing on the first motion control; and wherein the controller is further adapted to display a first lock indicator on the display when the first motion control is in the locked state and the user is not pressing on the first motion control, and wherein the controller is further adapted to display a second lock indicator on the display when the first motion control is in the locked state and the user is pressing on the first motion control.

2. The patient support apparatus of claim 1 wherein the controller is adapted to display both the first lock indicator and the second lock indicator on the display when the first motion control is in the locked state and the user is pressing on the first motion control.

3. The patient support apparatus of claim 2 wherein one of the first and second lock indicators is a lock symbol.

4. The patient support apparatus of claim 3 wherein the other one of the first and second lock indicators is a circle positioned around a perimeter of the first motion control.

5. The patient support apparatus of claim 1 wherein the controller is further adapted to fade the second lock indicator away after the user stops pressing on the first motion control.

6. The patient support apparatus of claim 1 wherein the controller is further adapted to display the first lock indicator adjacent the first motion control, and to display the second lock indicator adjacent the first motion control.

7. The patient support apparatus of claim 1 further comprising a second motion control, wherein the controller is adapted to switch the second motion control between the active state and the locked state, to activate the actuator in a first direction in response to the user pressing on the first motion control, and to activate the actuator in a second and opposite direction in response to the user pressing on the second motion control.

8. The patient support apparatus of claim 7 wherein the controller is further adapted to display a third lock indicator on the display when the second motion control is in the locked state and the user is not pressing on the first motion control, and to display a fourth lock indicator on the display when the second motion control is in the locked state and the user is pressing on the second motion control.

9. The patient support apparatus of claim 8 wherein the controller is adapted to display both the third lock indicator and the fourth lock indicator on the display when the second motion control is in the locked state and the user is pressing on the second motion control.

10. The patient support apparatus of claim 1 wherein the controller is further adapted to display a lock screen on the display after a period non-use of the control panel.

11. (canceled)

12. The patient support apparatus of claim 10 wherein the lock screen includes an unlocking control and the controller is further adapted to not allow the user to navigate to another screen until the user activates the unlock control, and wherein the lock screen includes no controls for controlling any movement of the patient support apparatus.

13. The patient support apparatus of claim 12 further comprising:
a litter frame adapted to support the support surface;
a tilt angle sensor adapted to sense a tilt angle of the litter frame; and
wherein the controller is adapted to display a litter frame tilt angle on the lock screen.

14. The patient support apparatus of claim 13 wherein the controller is adapted to display the litter frame tilt angle in a first manner when the litter frame tilt angle is zero degrees and in a second manner different from the first manner when the litter frame tilt angle is not zero degrees.

15. The patient support apparatus of claim 13 wherein the controller is further adapted to display a concealment control adapted to selectively conceal and reveal the litter frame tilt angle.

16. The patient support apparatus of claim 15 wherein the controller is further adapted to display a symbol when the litter frame tilt angle is concealed, to change a characteristic of the symbol when the litter frame tilt angle is non-zero, and to display a numeric value of the litter frame tilt angle when the litter frame tilt angle is not concealed.

17. The patient support apparatus of claim 15 wherein the lock screen includes no controls other than the concealment control and the unlock control.

18. The patient support apparatus of claim 17 wherein the unlock control requires the user to drag a slider icon on the display and thereafter continues to press down on the slider icon for a predetermined time period.

19. The patient support apparatus of claim 18 wherein the controller is adapted to display a lock icon on the lock screen and to animate unlocking movement of the lock icon as the user drags the slider icon on the display.

20. The patient support apparatus of claim 19 wherein the controller is further adapted to animate a circle being drawn around the slider icon when the user presses down on the slider icon for the predetermined time period.

21. The patient support apparatus of claim 20 wherein the controller is further adapted to finish animating the circle being drawn around the slider icon upon expiration of the predetermined time period.

22-99. (canceled)
